(19) United States
(12) Patent Application Publication
(10) Pub. No.: US 2016/0007838 A1
(43) Pub. Date: Jan. 14, 2016

(54) MEDICAL DEVICE

(71) Applicant: TERUMO KABUSHIKI KAISHA, Tokyo (JP)

(72) Inventors: Shigeki ARIURA, Ebina-shi (JP); Atsuhiko NOGAWA, Naka-gun Kanagawa (JP); Masakatsu KAWAURA, Sunnyvale, CA (US)

(73) Assignee: TERUMO KABUSHIKI KAISHA, Tokyo (JP)

(21) Appl. No.: 14/859,740

(22) Filed: Sep. 21, 2015

Related U.S. Application Data
(63) Continuation of application No. PCT/JP2014/057766, filed on Mar. 20, 2014.

(30) Foreign Application Priority Data
Mar. 22, 2013 (JP) 2013-061081

(51) Int. Cl. A61B 1/018; A61B 1/303

(52) U.S. Cl. CPC A61B 1/018; A61B 1/303; A61F 2002/0072

(57) ABSTRACT

A medical device is disclosed which connects an overactive tissue suffering from positional displacement in a living body to a less active tissue having a range of movement smaller than that of the overactive tissue to correct the positional displacement. The medical device can include an elongated insertion tool including an observation portion configured to observe a distal end side of the medical device using an imaging device, and a guide portion configured to guide a treatment tool, which is to be used to carry out treatment for the less active tissue, to the proximity of the less active tissue, the insertion tool having a dissection function for dissecting a biological tissue when the insertion tool is inserted into the living body from the outside of the living body and advances toward the less active tissue in the living body.

### CROSS-REFERENCES TO RELATED APPLICATIONS

This application is a continuation of International Application No. PCT/JP2014/057766 filed on Mar. 20, 2014, and claims priority to Japanese Application No. 2013-061081 filed on Mar. 22, 2013, the entire content of both of which is incorporated herein by reference.

### TECHNICAL FIELD

The present disclosure generally relates to a medical device.

### BACKGROUND DISCUSSION

As a disease peculiar to women, a disease called “pelvic organ prolapse (POP)” is known by which an organ located in the pelvis such as the uterus, bladder, urethra, small intestine or rectum drops into the vagina and prolapses from the female genitalia to the outside together with the vaginal wall due to damage to or weakening of a ligament, a fascia, a muscle or the like by which the vagina is supported. The “pelvic organ prolapse” is a generic name, and where the uterus prolapses, the disease may be called “uterine prolapse”; where an interior portion of the vagina prolapses in a state in which the uterus is removed, the disease may be called “vaginal vault prolapse”; where the bladder prolapses, the disease may be called “cystocele”; where the urethra prolapses, the disease may be called “urethra aneurysm”; where the small intestine prolapses, the disease may be called “small intestine aneurysm”; and where the rectum prolapses, the disease may be called “rectocele.”

As a surgical technique for such a pelvic organ prolapse, for example, a method is known wherein a string or a mesh is used to fix a prolapsed organ to a tissue whose range of motion with respect to the pelvis is small (for example, a ligament or a fascia in the proximity of the sacrum; hereinafter referred to also as “less active tissue.”). In such a surgical technique as just described, in order to fix the string to the less active tissue, an elongated suture appliance as disclosed, for example, in Japanese Patent No. 4125870 is used. However, according to the method, which uses the suture appliance described above, since it is necessary to blindly carry out a manipulation for fixing the string to the less active tissue, the string may not be able to be fixed appropriately to the less active tissue or a surrounding tissue may be damaged. Thus, in order to carry out a surgical procedure safely, skill of the operator is required.

### SUMMARY

A medical device is disclosed which is used in a surgical procedure, for example, in a surgical procedure of pelvic organ prolapse and is relatively high in safety and operability.

A medical device is disclosed which connects an overactive tissue suffering from positional displacement in a living body to a less active tissue having a range of movement smaller than that of the overactive tissue to correct the positional displacement, including an elongated insertion tool including an observation portion configured to observe a distal end side of the medical device using an imaging device, and a guide portion configured to guide a treatment tool, which is to be used to carry out treatment for the less active tissue, to proximity of the less active tissue, the insertion tool having a dissection function for dissecting a biological tissue when the insertion tool is inserted into the living body from an outside of the living body and advances toward the less active tissue in the living body.

The medical device may be configured such that the guide portion is a treatment tool lumen provided in an inside of the insertion tool

In accordance with an exemplary embodiment, the medical device may be configured such that the treatment tool lumen has a distal end side opening which is positioned at a distal end portion of the insertion tool.

Alternatively, the medical device may be configured such that the treatment tool lumen has a proximal end side opening which is positioned, in a state in which the insertion tool is inserted in the living body, outside the living body.

The medical device may be configured such that the treatment tool has an elongated article which has an elongated portion and an anchor portion provided on the elongated portion and configured to engage with the less active tissue.

In accordance with an exemplary embodiment, the medical device may be configured such that the treatment tool further has an operation tool configured to push the anchor portion to an outside of the insertion tool from the treatment tool lumen.

Further, the medical device may be configured such that the elongated article advances, in an insertion state in which the elongated article is inserted in the treatment tool lumen, toward the less active tissue together with the insertion tool.

Furthermore, the medical device may be configured such that, in the insertion state, the anchor portion projects through the distal end side opening of the treatment tool lumen.

Furthermore, the medical device may be configured such that, in the insertion state, retraction of the anchor portion into the treatment tool lumen is restricted.

Alternatively, the medical device may be configured such that the anchor portion is a linear article shaped along an outer periphery of the insertion tool.

Furthermore, the medical device may be configured such that the anchor portion is engaged with the less active tissue by operating the insertion tool to rotate in a state in which the anchor portion is introduced to the less active tissue.

The medical device described above can include an elongated insertion tool having an observation portion configured to observe a distal end side of the medical device using an imaging device, and a guide portion configured to guide a treatment tool, which can be used to carry out treatment for a less active tissue, to the proximity of the less active tissue, the insertion tool having a dissection function for dissecting a biological tissue when the insertion tool is inserted into the living body from the outside of the living body and advances toward the less active tissue in the living body. Therefore, with the medical device, insertion of the insertion tool can be carried out visibly while an image obtained from the imaging device is observed. Consequently, the guide portion can be disposed accurately at a predetermined position, and treatment for the less active tissue by the treatment tool can be carried out relatively safely and accurately.

A method is disclosed for treating an overactive tissue suffering from positional displacement in a living body, the method comprising: incising a portion of a vaginal wall prolapsed from a female genitalia to form an incision portion; inserting an elongated insertion tool on which a guide tool is mounted, through the incision portion, the elongated insertion tool including an observation portion configured to observe a distal end side of the medical device using an imaging device, and an indwelled portion configured to be indwelled in the living body in a state in which the indwelled portion engages a less active tissue; positioning a distal end of the insertion tool at the less active tissue; pulling out the insertion tool from the guide tool; inserting a treatment tool through a proximal end side opening of the guide tool and guiding the treatment tool to the less active tissue; carrying out a predetermined treatment for the less active tissue using the treatment tool; and correcting the positional displacement of the overactive tissue.

### BRIEF DESCRIPTION OF THE DRAWINGS

FIGS. 1A and 1B are lateral views of a medical device according to a first exemplary example in a mounted state and a dismounted state, respectively;

FIG. 2 is an enlarged sectional view of a distal end portion of an insertion tool, which includes the medical device depicted in FIGS. 1A and 1B;

FIG. 3 is a block diagram of an external apparatus connected to the medical device depicted in FIGS. 1A and 1B;

FIG. 4A is a lateral view depicting a treatment tool guided by a guide tool, which includes the medical device depicted in FIGS. 1A and 1B;

FIG. 4B is an enlarged sectional view of a distal end portion of the treatment tool;

FIGS. 5A and 5B are sectional views illustrating a movement of the treatment tool depicted in FIGS. 4A and 4B;

FIGS. 6A and 6B are schematic sectional views illustrating steps of a surgical procedure in which the medical device depicted in FIGS. 1A and 1B is used;

FIGS. 7A and 7B are schematic sectional views illustrating steps of a surgical procedure in which the medical device depicted in FIGS. 1A and 1B is used;

FIGS. 8A and 8B are schematic sectional views illustrating steps of a surgical procedure in which the medical device depicted in FIGS. 1A and 1B is used;

FIGS. 9A and 9B are schematic sectional views illustrating steps of a surgical procedure in which the medical device depicted in FIGS. 1A and 1B is used;

FIGS. 10A and 10B are a lateral view and a sectional view, respectively, of an insertion tool of a medical device according to a second exemplary example;

FIG. 10C is a sectional view of the insertion tool in which an endoscope is inserted;

FIGS. 11A and 11B are lateral views depicting a medical device according to a third exemplary example in a mounted state and a dismounted state, respectively;

FIG. 12A is a lateral view depicting a treatment tool guided by a guide tool, which includes the medical device depicted in FIGS. 11A and 11B:

FIG. 12B is a lateral view, partly in section, of the treatment tool guided by the guide tool;

FIG. 13A is a schematic view depicting an implant, which includes the guide tool depicted in FIGS. 12A and 12B;

FIG. 13B is a schematic view depicting the implant in a state in which it is held by a holding portion;

FIGS. 14A and 14B are schematic sectional views illustrating steps of a surgical procedure in which the medical device depicted in FIGS. 11A and 11B is used;

FIGS. 15A and 15B are schematic sectional views illustrating steps of a surgical procedure in which the medical device depicted in FIGS. 11A and 11B is used;

FIGS. 16A and 16B are schematic sectional views illustrating steps of a surgical procedure in which the medical device depicted in FIGS. 11A and 11B is used;

FIG. 17 is a schematic sectional view illustrating step of a surgical procedure in which the medical device depicted in FIGS. 11A and 11B is used;

FIGS. 18A and 18B are lateral views depicting a medical device according to a fourth exemplary example in a mounted state and a dismounted state, respectively;

FIGS. 19A and 19B are sectional views illustrating a movement of the medical device depicted in FIGS. 18A and 18B;

FIGS. 20A and 20B are schematic sectional views illustrating steps of a surgical procedure in which the medical device depicted in FIGS. 18A and 18B is used;

FIGS. 21A and 21B are schematic sectional views illustrating steps of a surgical procedure in which the medical device depicted in FIGS. 18A and 18B is used;

FIGS. 22A and 22B are schematic sectional views illustrating steps of a surgical procedure in which the medical device depicted in FIGS. 18A and 18B is used;

FIGS. 23A and 23B are a lateral view and a sectional view, respectively, depicting a medical device according to a first embodiment of the present disclosure;

FIG. 24A is a schematic view depicting a treatment tool guided by the medical device depicted in FIGS. 23A and 23B;

FIGS. 24B and 24C are sectional views illustrating a guiding method for the treatment tool;

FIGS. 25A and 25B are schematic sectional views illustrating steps of a surgical procedure in which the medical device depicted in FIGS. 23A and 23B is used;

FIGS. 26A and 26B are schematic sectional views illustrating steps of a surgical procedure in which the medical device depicted in FIGS. 23A and 23B is used;

FIGS. 27A and 27B are schematic sectional views illustrating steps of a surgical procedure in which the medical device depicted in FIGS. 23A and 23B is used;

FIGS. 28A and 28B are a lateral view and a sectional view, respectively, depicting a medical device according to a second embodiment of the present disclosure;

FIG. 29A is a schematic view depicting a treatment tool which is guided by the medical device depicted in FIGS. 28A and 28B;

FIGS. 29B and 29C are a sectional view and a front view, respectively, depicting an insertion state of the treatment tool;

FIGS. 30A and 30B are schematic sectional views illustrating steps of a surgical procedure in which the medical device depicted in FIGS. 28A and 28B is used;

FIGS. 31A and 31B are schematic sectional views illustrating steps of a surgical procedure in which the medical device depicted in FIGS. 28A and 28B is used;

FIG. 32 is a schematic sectional view illustrating steps of a surgical procedure in which the medical device depicted in FIGS. 28A and 28B is used;

FIG. 33 is a schematic view depicting a treatment tool, which is guided by the medical device according to the second embodiment of the present disclosure;

FIGS. 34A and 34B are schematic sectional views illustrating steps of a surgical procedure in which the medical device according to a third embodiment of the present disclosure is used;

FIGS. 35A and 35B are schematic sectional views illustrating steps of a surgical procedure in which the medical device according to the third embodiment is used; and

FIG. 36 is a schematic sectional view illustrating steps of a surgical procedure in which the medical device according to the third embodiment is used.

### DETAILED DESCRIPTION

In the following, a medical device of the present disclosure is described in detail in connection with preferred embodiments of the present disclosure depicted in the accompanying drawings.

First, a medical device according to a first exemplary example is described.

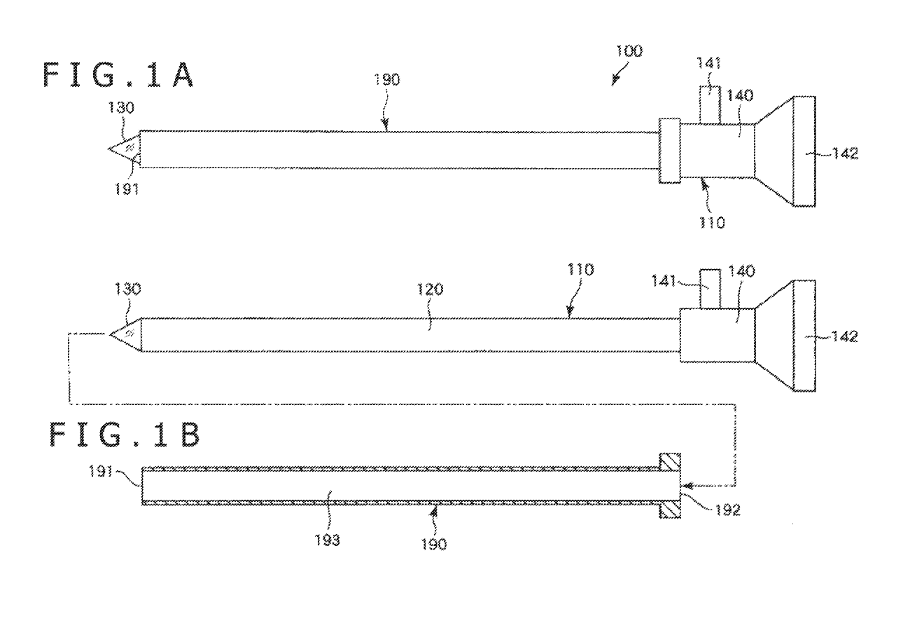

It is to be noted that, in the following description, the left side and the right side in FIGS. 1A to 2 and 4A to 5B are referred to as “distal end” and “proximal end,” respectively, for the convenience of description.

FIGS. 1A and 1B depict a medical device 100 which is a device to be used for treatment of pelvic organ prolapse (POP), for example, to be used to return an organ, which is prolapsed from the female genitalia together with the vaginal wall, to its normal position (to correct the organ against positional displacement).

It is to be noted that, in the following description, an organ which suffers from positional displacement from among organs positioned in the pelvis such as the vagina, uterus, bladder, urethra, small intestine and rectum is referred to sometimes as “overactive tissue” and a tissue whose range of motion with respect to the pelvis is smaller than that of the overactive tissue is referred to sometimes as “less active tissue” for the convenience of description. Such less active tissues include, for example, bones (pelvis) such as hipbones, sacra and coccyx, ligaments such as sacrospinous ligaments, anterior sacrococcygeal ligaments, sacrotuberous ligaments and anterior sacro-iliac ligaments, tendons, fascias, muscle tissues and so forth.

As depicted in FIG. 1A, the medical device 100 can include an insertion tool 110 of an elongated shape to be inserted into a living body, and a guide tool 190 of a tubular shape which is removably mounted on the insertion tool 110. In the following, the insertion tool 110 and the guide tool 190 are successively described in detail.

First, the insertion tool 110 is described. Referring to FIG. 1B, the insertion tool 110 can include a main body 120 in the form of a bar, an observation portion 130 provided at a distal end portion of the main body 120, and a connector portion 140 provided at a proximal end portion of the main body 120. The constituent materials of the main body 120, the observation portion 130 and the connector portion 140 are not limited particularly, and, for example, various resin materials can be used for them.

The main body 120 can have a form of a bar extending straight. Although the length of the main body 120 is not limited particularly, for example, it preferably is approximately 10 cm to 30 cm. This provides the insertion tool 110 with a length suitable for medical treatment of pelvic organ prolapse.

The observation portion 130 is provided at the distal end portion of the main body 120. Further, the observation portion 130 is substantially colorless and transparent and has a light permeability. Further, the observation portion 130 has a vertex of a substantially conical shape directed toward the distal end side. In accordance with an exemplary embodiment, the observation portion 130 tapers toward the distal end of the insertion tool 110. Since the observation portion 130 has a light permeability and has a tapering shape toward the distal end of the insertion tool 110, when the insertion tool 110 advances toward an object region in a living body, the observation portion 130 exhibits a function for dissecting the biological tissue while avoiding a thick blood vessel in the biological tissue. Consequently, insertion of the insertion tool 110 into the living body can be carried out minimally invasively and relatively smoothly. It is to be noted that the shape of the observation portion 130 is not limited to such a conical shape as that in the present exemplary embodiment and may be, for example, a tapering shape (duckbill shape) such that the distal end thereof of the observation portion 130 has a linear shape. Further, the observation portion 130 is not limited to that of a colorless and transparent type and may be colored to red, blue, or green if the observation portion 130 has a light permeability.

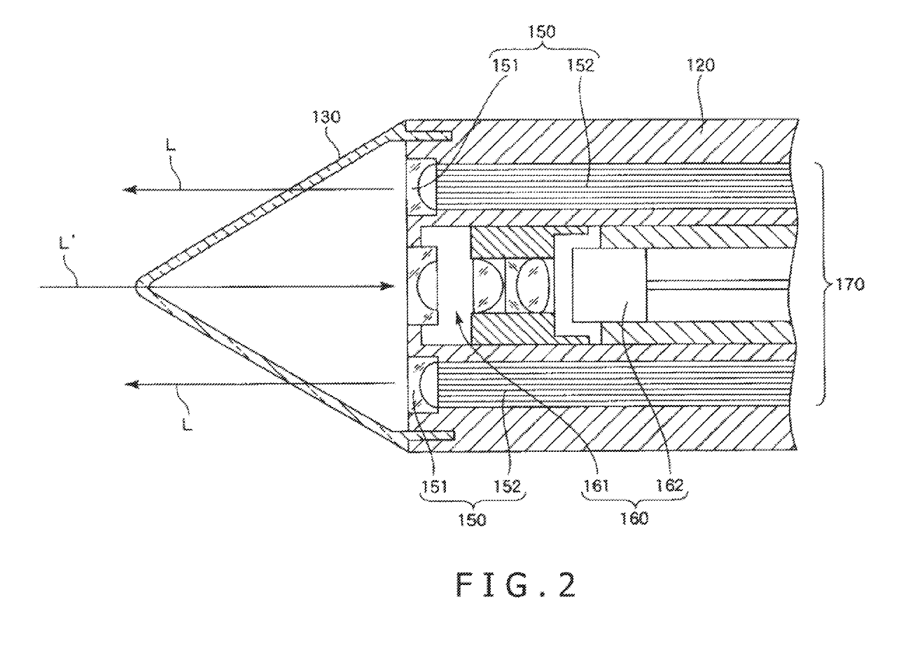

Further, as depicted in FIG. 2, an imaging device 170 is disposed in the inside of the main body 120. The imaging device 170 can include an illumination unit 150 which emits illumination light for illuminating forwardly of the insertion tool 110 (to the distal end side), and an image pickup unit 160 which images forwardly of the insertion tool 110. The illumination unit 150 can include a lens 151 disposed at a distal end portion of the main body 120 and an optical fiber bundle 152 provided behind the lens 151. Meanwhile, the image pickup unit 160 can include an objective lens system 161 provided at a distal end portion of the main body 120, and an image pickup element (solid-state image pickup element such as, for example, a complementary metal oxide semiconductor (CMOS) image sensor or a charge coupled device (CCD) sensor) 162 disposed in an opposing relationship to the objective lens system 161.

The connector portion 140 has an increased diameter from that of the main body 120 as depicted in FIG. 1B and functions as a stopper for the guide tool 190 mounted on the insertion tool 110. Further, the connector portion 140 can include a light source connector 141 connected to the proximal end of the optical fiber bundle 152 and an image signal connector 142 connected to the image pickup element 162. The insertion tool 110 is connectable to an external apparatus 900 through the light source connector 141 and the image signal connector 142.

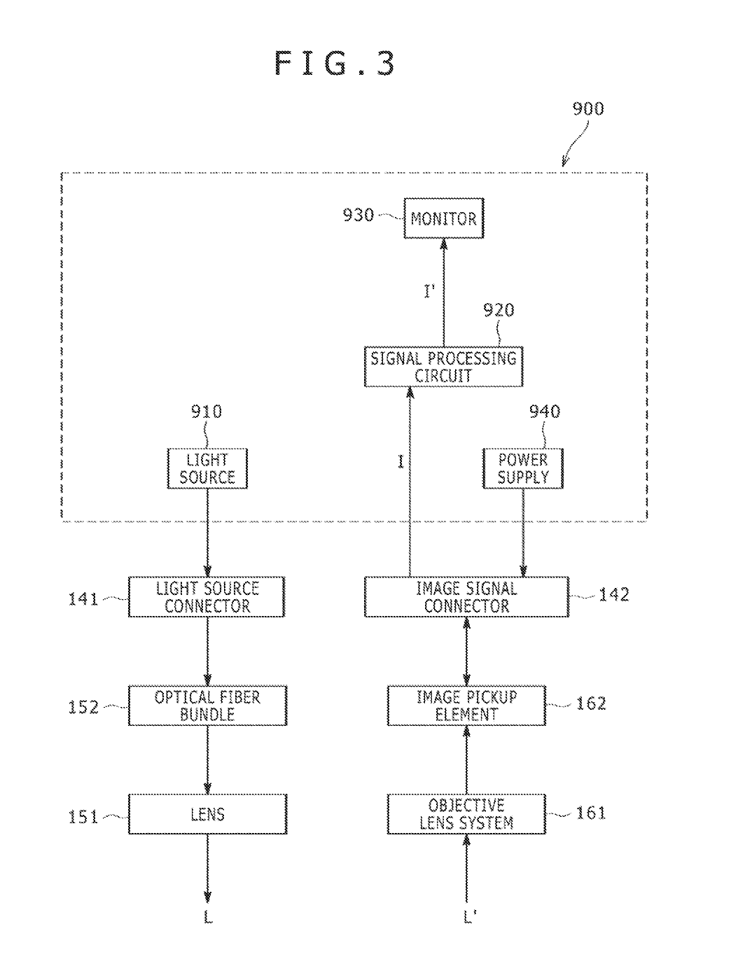

As depicted in FIG. 3, the external apparatus 900 can include a power supply 940 which supplies power to the image pickup element 162 through the image signal connector 142 and enables driving of the image pickup element 162. Further, the external apparatus 900 can include a light source 910 and emits illumination light L from the light source 910 simultaneously when power is supplied to the image pickup element 162. The illumination light L is introduced into the optical fiber bundle 152 through the light source connector 141, passes through the optical fiber bundle 152 and is emitted from the lens 151. The emitted illumination light L passes through the observation portion 130 and is irradiated forwardly of the insertion tool 110. The illumination light L irradiated forwardly becomes reflected light L′ which is to form an image pickup object image, passes through the observation portion 130 and enters the objective lens system 161, by which it is introduced to a light receiving face of the image pickup element 162. Then, as the image pickup element 162 receives the reflected light L′, the image pickup object image is picked up and an image signal I corresponding to the image pickup object image is outputted from the image pickup element 162. The image signal I is inputted to a signal processing circuit 920 provided in the external apparatus 900 through the image signal connector 142 and is converted into a predetermined television signal I′ by the signal processing circuit 920. The television signal I′ is inputted to a monitor 930, and an image (moving picture or still picture) of the image pickup object picked up by the image pickup element 162 is display on the screen of the monitor 930.

Now, the guide tool 190 is described. The guide tool 190 has a function of guiding a treatment tool 800 to a target region (less active tissue) by being indwelled in a living body after guide tool 190 is inserted into the living body together with the insertion tool 110.

As depicted in FIG. 1B, the guide tool 190 has a form of elongated and substantially straight cylinder. Therefore, the guide tool 190 has a distal end side opening 191, a proximal end side opening 192, and a through-hole 193 which connects the distal end side opening 191 and the proximal end side opening 192 to each other. The guide tool 190 is mounted on the insertion tool 110 as depicted in FIG. 1A by inserting (fitting) the insertion tool 110 into the through-hole 193 through the proximal end side opening 192. For the convenience of description, the state just described is hereinafter referred to as “mounted state.”

The guide tool 190 has a length substantially equal to that of the main body 120. Therefore, in the mounted state, the observation portion 130 projects through the distal end side opening 191 of the guide tool 190 as depicted in FIG. 1A. Therefore, the observation portion 130 can exhibit a dissection function with certainty without being obstructed by the guide tool 190, and image pickup by the image pickup unit 160 can be carried out with certainty. It is to be noted that, although the length of the guide tool 190 is not limited particularly, for example, the guide tool 190 is preferably as long as possible unless it covers the observation portion 130 in the mounting state. Since this makes it possible to position the distal end of the guide tool 190 nearer to a target region, the treatment tool 800 can be guided to the target region with a high degree of certainty.

Although the inner diameter of the guide tool 190 is not limited particularly, for example, the guide tool 190 preferably is equal to or a little greater than the outer diameter of the main body 120, which makes it possible to suppress looseness of the guide tool 190 with respect to the main body 120 and improves the operability of the medical device 100. Further, although the outer diameter of the guide tool 190 is not limited particularly, for example, it preferably is approximately 5 mm to 20 mm, which helps provides the medical device 100 with less invasiveness and a good operability.

The constituent material for such a guide tool 190 as described above is not limited particularly, and, for example, various resin materials and various metal materials can be used.

It is to be noted that, although the guide tool 190 in the present exemplary embodiment has a tubular shape in an overall range thereof, the shape of the guide tool 190 is not limited particularly if the guide tool 190 can guide the treatment tool 800. For example, a semi-tubular portion as may be obtained by dividing a tube to half may be provided at part of the guide tool 190 in the extension direction. Even with such a shape as just described, an effect similar to that achieved by the present exemplary embodiment can be achieved.

The medical device 100 has been described. In the present exemplary embodiment, an electronic device including the image pickup element 162 is used as the imaging device 170. However, the configuration of the imaging device 170 is not limited particularly if the imaging device 170 can view (image) forwardly of the insertion tool 110. For example, the imaging device 170 may be an optical imaging device configured such that a lens is disposed at a distal end portion of the main body 120 while an eyepiece is disposed on the connector portion 140 and an optical fiber bundle interconnects the lens and the eyepiece such that light passing through the lens is propagated by the optical fiber and forms an image through the eyepiece. Alternatively, the imaging device 170 may be configured such that an ultrasonic device including an ultrasonic wave transmission and reception device is disposed at a distal end portion of the main body 120 such that an ultrasonic image can be formed on the basis of a signal obtained from the ultrasonic wave transmission and reception device.

Now, the treatment tool 800 which is guided to a target region by the guide tool 190 is described briefly.

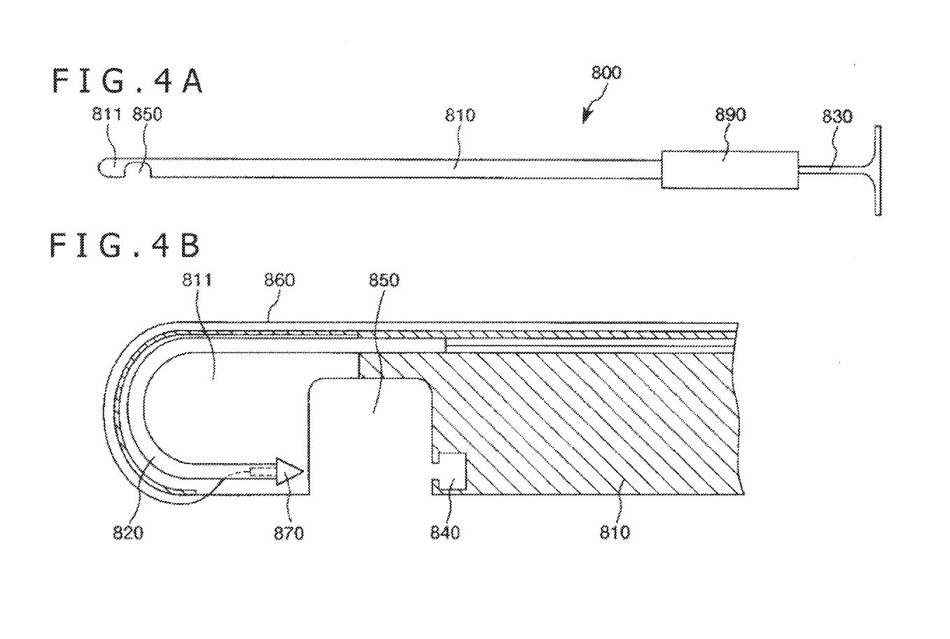

The treatment tool 800 is a device for carrying out treatment for a less active tissue. As depicted in FIGS. 4A and 4B, the treatment tool 800 can include an elongated main body 810, a needle carrier 820 provided at a distal end portion of the main body 810 and holding and transporting a needle 870 having a string (elongated article) 860 connected thereto, and an operation lever 830 provided on the proximal end side of the main body 810 for operating the needle carrier 820. The treatment tool 800 further can include a needle catch 840 provided on the main body 810 for catching the needle 870 transported by the needle carrier 820, and a stopper 890 provided at a proximal end portion of the main body 810. The string 860 is configured from various resin materials and textiles having a biocompatibility like polypropylene.

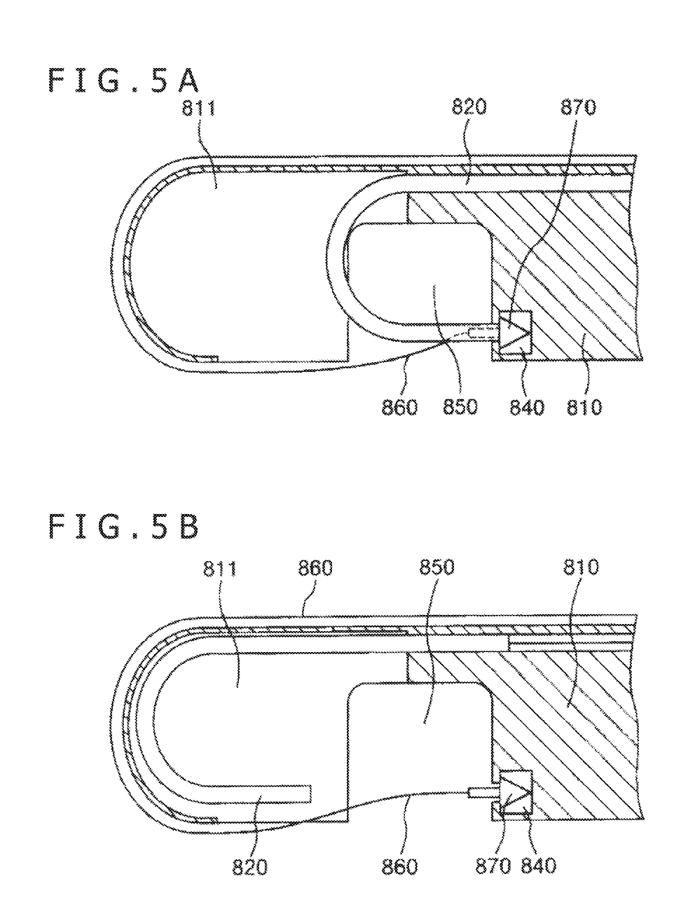

The main body 810 has, at a distal end portion thereof, an accommodation portion 811 for accommodating the needle carrier 820 therein. If the operation lever 830 is operated, then the needle carrier 820 can be projected from the accommodation portion 811 toward the needle catch 840 or can be retracted into the accommodation portion 811. The needle catch 840 is provided on the proximal end side of the accommodation portion 811 in a spaced relationship from the accommodation portion 811 with a space 850 interposed therebetween. If the needle carrier 820 is projected from the accommodation portion 811 as depicted in FIG. 5A, then the needle 870 held on the needle carrier 820 is caught by the needle catch 840. Since this state is maintained, if the needle carrier 820 is retracted into the accommodation portion 811 thereafter, then the string 860 is placed into a state in which it is laid bypassing the space 850 as depicted in FIG. 5B. Therefore, by operating the treatment tool 800 in a state in which a target on which the string 860 is to be hooked is disposed in the space 850, the string 860 can be hooked simply on the target.

Such a treatment tool 800 as described above is configured such that, if the main body 810 is inserted into the guide tool 190 through the proximal end side opening 192, then the stopper 890 is abutted with the proximal end side opening 192 so that it is restricted from being inserted further. In the state in which the stopper 890 abuts with the proximal end side opening 192, the space 850 projects through the distal end side opening 191 of the guide tool 190. Consequently, excessive projection of the treatment tool 800 from the guide tool 190 can be prevented, and treatment for a less active tissue can be carried out relatively safely and with relative certainty.

It is to be noted that the configuration of the treatment tool is not limited particularly only if it is configured such that it allows the string 860 to be hooked on the target.

Now, usage of the medical device 100 is described. It is to be noted that, in the following description, a surgical treatment of vaginal vault prolapse which is a kind of pelvic organ prolapse is taken as an example.

A surgical procedure in which the medical device 100 is used can include a step of incising part of a vaginal wall H11 prolapsed from the female genitalia to form an incision portion H111, a step of inserting the insertion tool 110, on which the guide tool 190 is mounted, through the incision portion H111 and positioning the distal end of the insertion tool 110 at a sacrospinous ligament (less active tissue) H2, a step of pulling out the insertion tool 110 from the guide tool 190, a step of inserting the treatment tool 800 through the proximal end side opening 192 of the guide tool 190 and guiding the treatment tool 800 to the sacrospinous ligament H2, a step of carrying out predetermined treatment for the sacrospinous ligament H2 using the treatment tool 800, and a step of correcting the positional displacement of a vagina (overactive tissue) H1. In the following, detailed description is given.

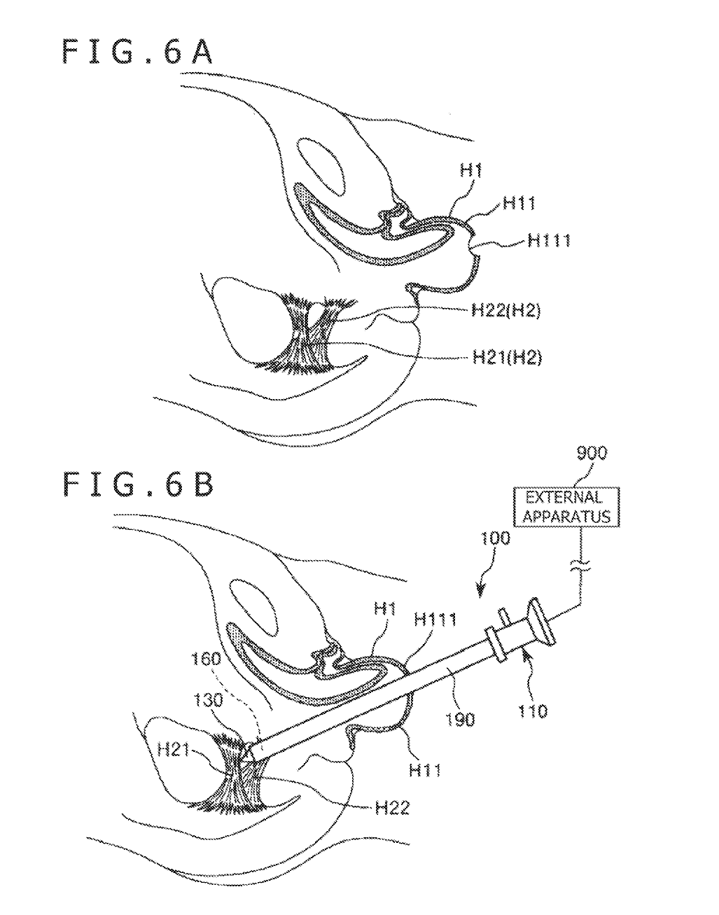

First, the vaginal wall H11 prolapsed from the female genitalia is incised to form the incision portion H111 as depicted in FIG. 6A. Then, the insertion tool 110 connected to the external apparatus 900 and having the guide tool 190 mounted on the insertion tool 110 is inserted into the living body through the incision portion H111 and advanced toward a left side sacrospinous ligament H21 as depicted in FIG. 6B. Since the observation portion 130 has a function for dissecting a biological tissue as described hereinabove, the insertion tool 110 can be advanced relatively smoothly. Further, since an image picked up by the image pickup unit 160 can be confirmed on the real time on the monitor, the insertion tool 110 can be advanced relatively accurately and safely.

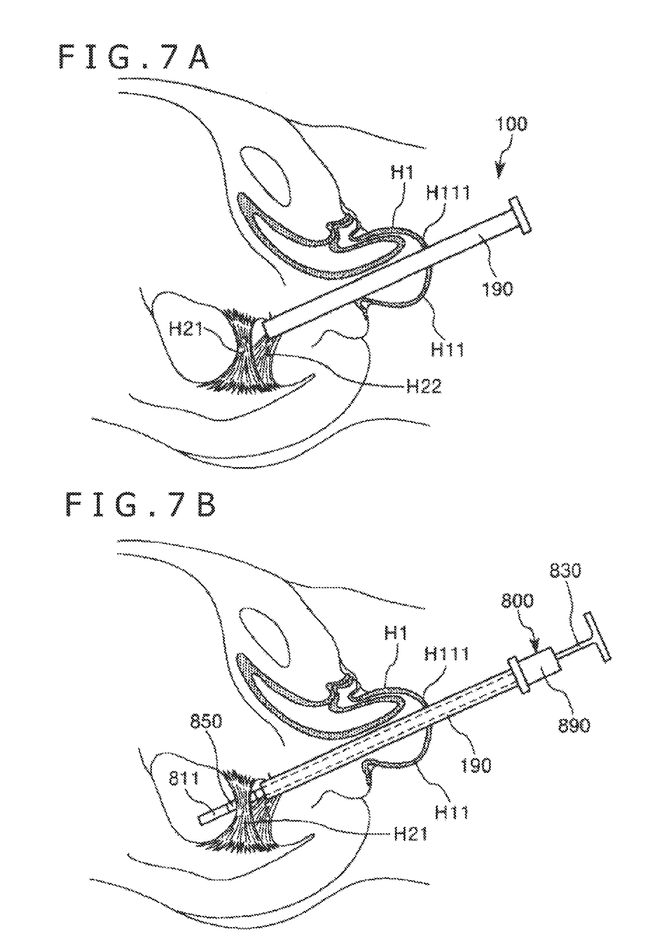

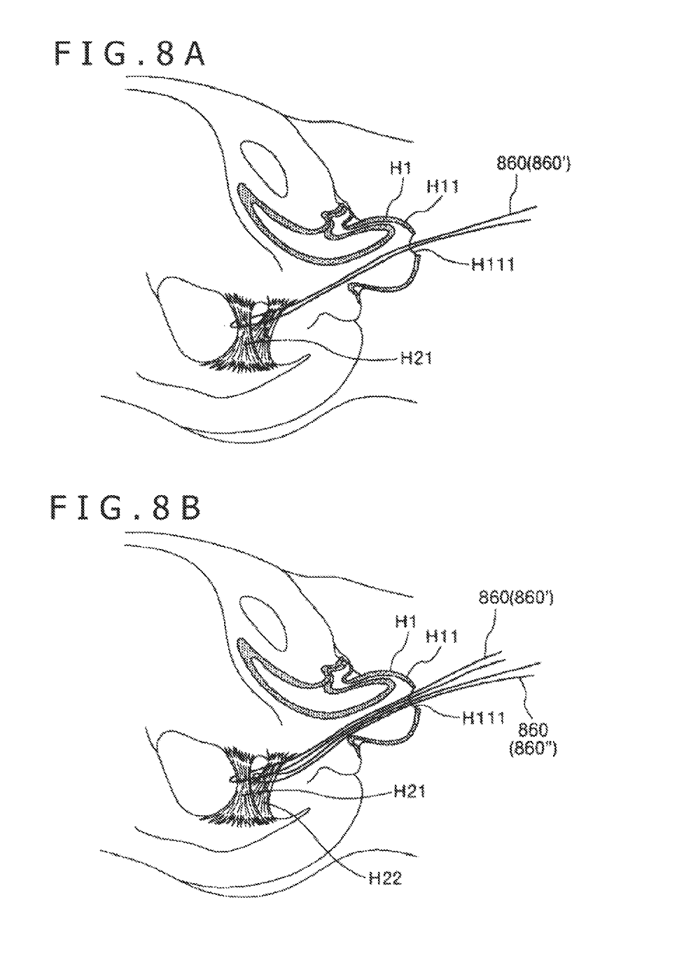

After the distal end of the insertion tool 110 arrives at the sacrospinous ligament H2 of the patient on the right side (in FIGS. 6A to 9B, 14A to 17, 20A to 22B, 25A to 27B, 30A to 32 and 34A to 36, the patient is in a face-up state (lithotomy position), and therefore, the right side of the patient corresponds to this side of the figures) or the proximity of the right side sacrospinous ligament H2, the insertion tool 110 is pulled out from the guide tool 190 to establish a state in which the guide tool 190 is indwelled in the living body as depicted in FIG. 7A. Then, a treatment tool 800 is inserted into the guide tool 190 through the proximal end side opening 192 and is guided to the sacrospinous ligament H21 by the guide tool 190. Then, the sacrospinous ligament H21 is disposed into the space 850 and the treatment tool 800 is operated to hook the string 860 on the sacrospinous ligament H21 as depicted in FIG. 7B. Thereafter, the treatment tool 800 is pulled out from the guide tool 190 and then the guide tool 190 is pulled out from the living body. Consequently, the opposite ends of the string 860 hooked on the sacrospinous ligament H21 are placed in a state in which they are exposed to the outside of the body through the incision portion H111 as depicted in FIG. 8A. The hooking of the string 860 on the sacrospinous ligament H21 is completed therewith.

Then, also the string 860 is hooked also on a left side sacrospinous ligament H22 of the patient by a procedure similar to that of the method described above. Consequently, the opposite ends of the string 860 (860′) hooked on the sacrospinous ligament H21 and the opposite ends of the string 860 (860″) hooked on the sacrospinous ligament H22 are placed into a state in which they are exposed to the outside of the body through the incision portion H111 as depicted in FIG. 8B.

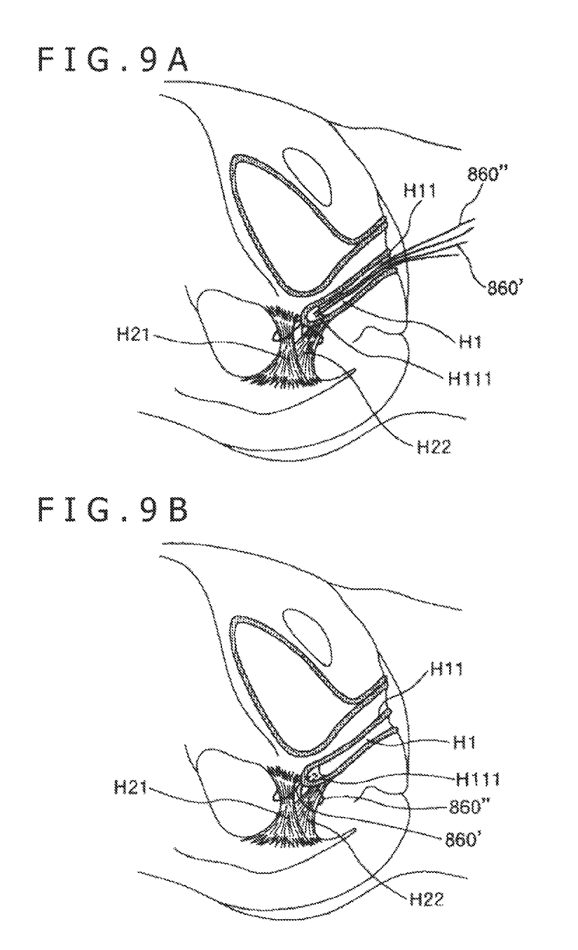

Then, the prolapsed vaginal wall H11 is returned to its normal position and the strings 860′ and 860″ are sutured to the vaginal wall H11 so that the state of the vaginal wall H11 is maintained, whereafter excessive portions of the strings 860′ and 860″ are cut away as depicted in FIG. 9A. Consequently, the sacrospinous ligaments H21 and H22 and the vaginal wall H11 are connected to each other by the strings 860′ and 860″, respectively, and the vagina H1 is corrected to the normal position in the female genitalia as depicted in FIG. 9B. Finally, the incision portion H111 is sutured and necessary treatments such as disinfection are carried out, thereby ending the surgical procedure.

In this manner, with the medical device 100, since insertion of the insertion tool 110 can be carried out visibly, the insertion tool 110 can be operated to reach a sacrospinous ligament H2 safely and with certainty. Therefore, the guide tool 190 is indwelled relatively accurately in the living body, and the treatment tool 800 can be guided accurately to the sacrospinous ligament H2 by the guide tool 190. As a result, hooking of the string 860 on the sacrospinous ligament H2 can be carried out with relative certainty and sufficiently. Therefore, the positional displacement of the vagina H1 can be cancelled to correct the position of the vagina H1 to its normal position relatively safely and with relative certainty.

It is to be noted that, while, in the surgical procedure described above, the strings 860 are individually hooked on the left and right sacrospinous ligaments H21 and H22, a string 860 may be hooked only on one of the sacrospinous ligaments. Further, while, in the surgical procedure described above, the strings 860′ and 860″ are hooked on the left and right sacrospinous ligaments H21 and H22 first and then the two strings 860′ and 860″ are sutured to the vaginal wall, the string 860′ may be hooked on the sacrospinous ligament H21 and then sutured to the vaginal wall H11, whereafter the string 860″ is hooked on the sacrospinous ligament H22 and sutured to the vaginal wall H11.

It sometimes occurs that the rectum is positioned on a pass route when the insertion tool 110 is advanced from the incision portion H111 toward the sacrospinous ligament H2 or in the proximity of the pass route. Therefore, the rectum may be damaged by the insertion tool 110. Therefore, in order to prevent damage to the rectum, such a procedure may be adopted that a bar-shaped rectum insertion tool is inserted into the rectum and then is operated to establish a state in which the position of the rectum is spaced sufficiently from the pass route of the insertion tool 110, whereafter, while this state is maintained, the insertion tool 110 is advanced toward the sacrospinous ligament H2. By the procedure, damage to the rectum by the insertion tool 110 can be prevented with certainty. It is to be noted that the method in which a rectum insertion tool is used can be applied similarly also to other exemplary embodiments hereinafter described.

Further, while the surgical procedure described above can include the step of hooking a string 860 on a sacrospinous ligament H2, this step may be carried out visibly. In accordance with an exemplary embodiment, for example, in the state in which the guide tool 190 is disposed in the living body (state of FIG. 7A), an endoscope of a small thickness with which it does not obstruct use of the treatment tool 800 may be inserted into the guide tool 190 together with the treatment tool 800 such that hooking of the string 860 on the sacrospinous ligament H2 is carried out while the sacrospinous ligament H2 is observed using the endoscope. This makes it possible to carry out hooking of the string 860 on the sacrospinous ligament H2 relatively safely and with relative certainty.

Further, while, in the surgical procedure described above, the strings 860′ and 860″ are hooked on the sacrospinous ligaments H2 as less active tissues, the less active tissues on which the strings 860′ and 860″ are to be hooked are not limited particularly only if they have a smaller range of motion with respect to the pelvis than the vagina H1. Such tissues include, for example, bones such as hipbones, sacra and coccyx (pelvis), tendons, ligaments other than sacrospinous ligaments such as anterior sacrococcygeal ligaments, sacrotuberous ligaments and anterior sacro-iliac ligaments, fascias such as obturator fascias which cover an obturator foramen and muscle tissues. Even if the string 860′ or 860″ is hooked on any of the tissues listed above, an effect similar to that achieved by the present exemplary embodiment can be achieved.

Further, while the overactive tissue is the vagina, since the surgical procedure described above is a surgical procedure of vaginal vault prolapse, the overactive tissue is not limited particularly. As the overactive tissue, the uterus, bladder, urethra, small intestine, large intestine, colon, recta and so forth are listed in addition to the vagina. The medical device 100 can be used similarly for treatment of such overactive tissues as just mentioned (surgical procedure of pelvic organ prolapse other than vaginal vault prolapse such as uterine prolapse, cystocele, urethra aneurysm, small intestine aneurysm and rectocele), and the positional displacement can be corrected similarly.

Now, a second exemplary example of a medical device is described.

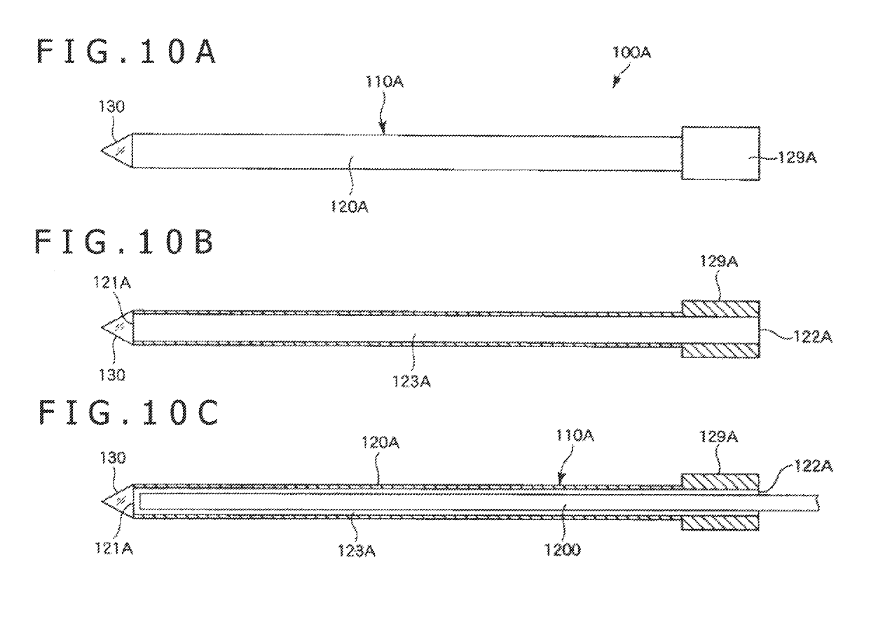

FIG. 10A is a lateral view of an insertion tool of the medical device according to the second exemplary example; FIG. 10B is a sectional view of the insertion tool; and FIG. 10C is a sectional view of the insertion tool in which an endoscope is inserted. It is to be noted that, in the following description, the left side in FIGS. 10A to 10C is referred to as “distal end” and the right side is referred to as “proximal end” for the convenience of description.

While the medical device of the present exemplary embodiment is described below, description is given principally of differences thereof from the medical device of the first exemplary example while description of similar matters is omitted herein to avoid redundancy.

The medical device of the present exemplary embodiment is similar to the medical device of the first exemplary example except that it is difference only in the configuration of the insertion tool.

As depicted in FIGS. 10A and 10B, in a medical device 100A of the present exemplary embodiment, the insertion tool 110A can include a main body 120A, and an observation portion 130 provided at a distal end portion of the main body 120A. The main body 120A has an elongated and substantially straight tubular shape and has a distal end side opening 121A, a proximal end side opening 122A and a through-hole 123A which connects the distal end side opening 121A and the proximal end side opening 122A to each other. Further, a stopper portion 129A is provided at a proximal end portion of the main body 120A, and has an increased diameter from that of the distal end side and functions as a stopper for a guide tool 190.

The distal end side opening 121A is covered with the observation portion 130. In the main body 120A of such a configuration as just described, an endoscope 1200 can be inserted into the through-hole 123A through the proximal end side opening 122A. By using the medical device 100A in the state in which the endoscope 1200 is inserted in the main body 120A, the medical device 100A can be used similarly to the medical device 100 of the first exemplary example described hereinabove. The endoscope 1200 may be, for example, an electronic endoscope which can include an image pickup device such as a CMOS image sensor or a CCD sensor or an optical endoscope by which light propagating along an optical fiber bundle forms an image by an eyepiece. Alternatively, instead of the endoscope 1200, an ultrasonic device including an ultrasonic transmission and reception apparatus may be inserted into the main body 120A such that the medical device 100A is used while the main body 120A visualizes a sight in front of the insertion tool 110A into an ultrasonic image.

Now, a third exemplary example of a medical device is described.

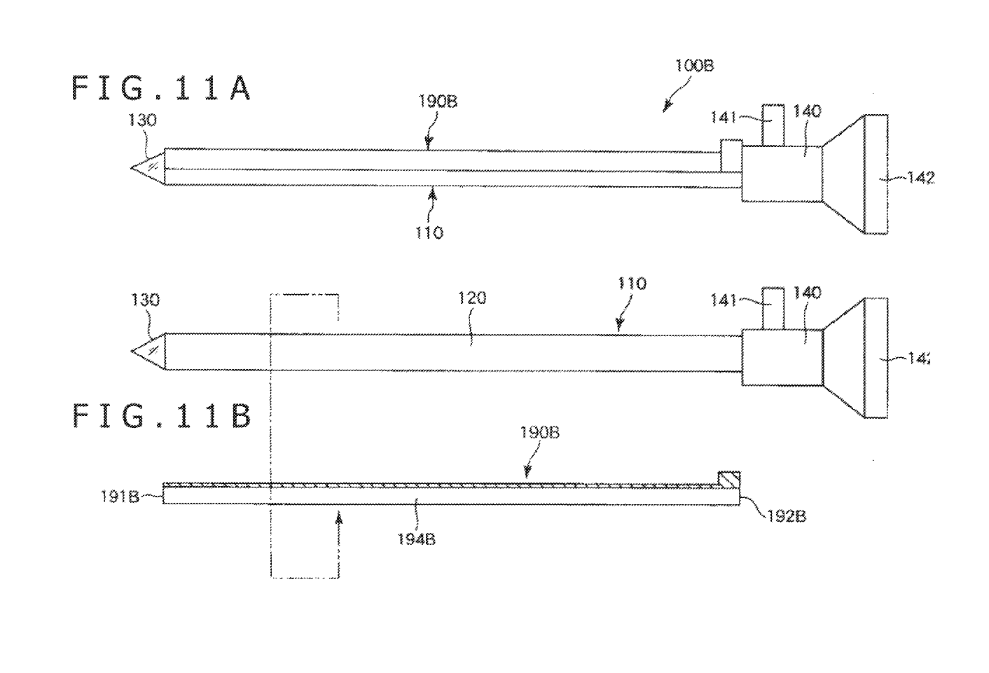

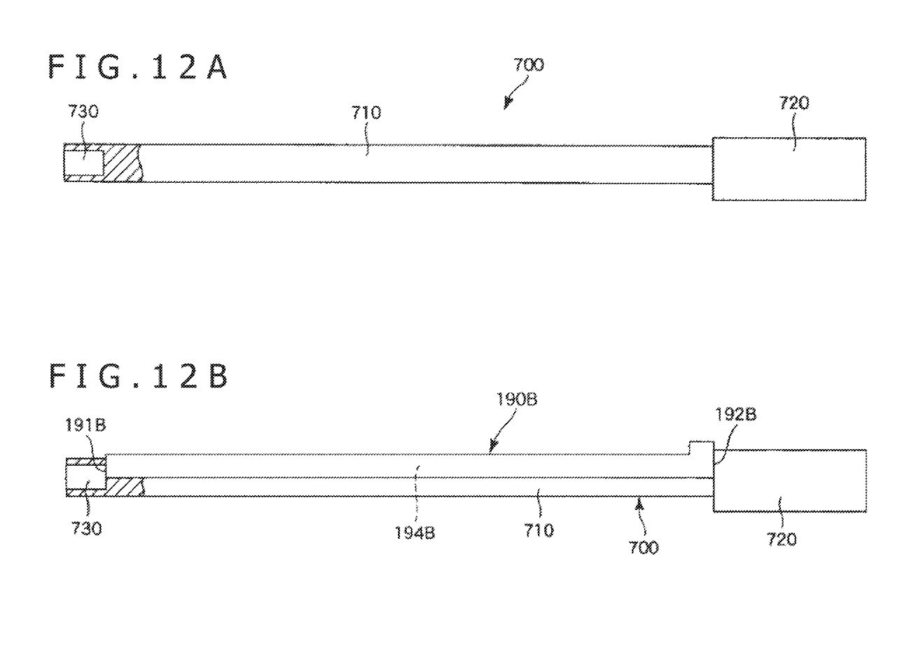

It is to be noted that, in the following description, the left side in FIGS. 11A to 12B is referred to as “distal end” and the right side is referred to as “proximal end” for the convenience of description.

While the medical device of the present exemplary embodiment is described below, description is given principally of differences thereof from the medical device of the first exemplary example while description of similar matters is omitted herein to avoid redundancy.

The medical device of the present exemplary embodiment is similar to the medical device of the first exemplary example except that it is different only in the configuration of the guide tool.

As depicted in FIGS. 11A and 11B, a guide tool 190B which a medical device 100B has is configured from such a semi-circular tube as obtained by dividing a cylinder to half. Therefore, the guide tool 190B has a distal end side opening 191B, a proximal end side opening 192B and a groove 194B which connects the distal end side opening 191B and the proximal end side opening 192B to each other. Such a guide tool 190B as just described is mounted on the insertion tool 110 through the groove 194B.

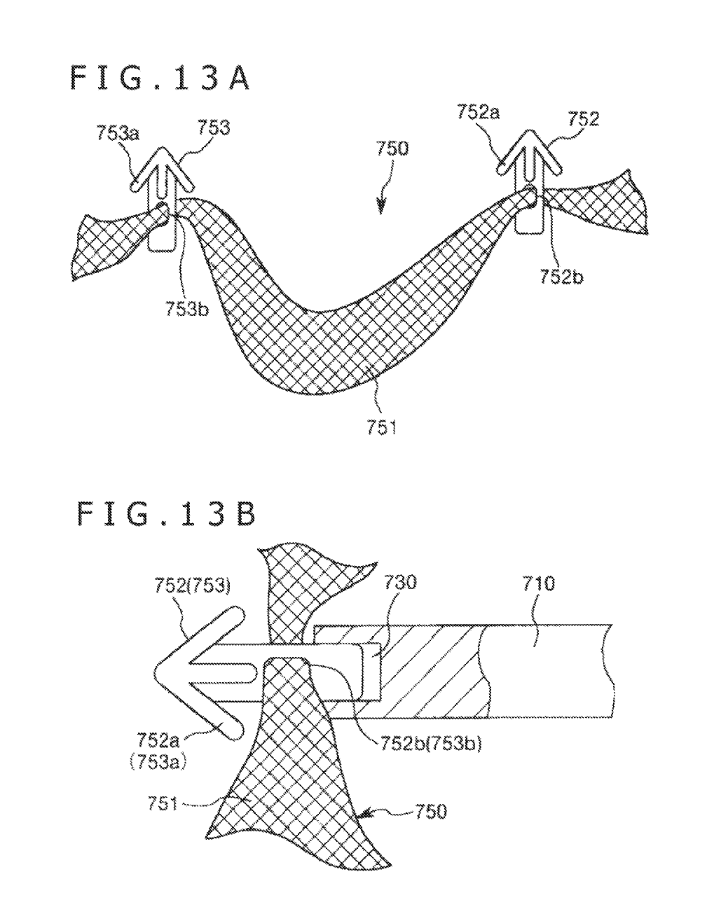

A treatment tool 700 is a tool for carrying out treatment for a less active tissue. As depicted in FIG. 12A, the treatment tool 700 can include a main body 710 of a shape of a bar extending substantially straightly, a stopper 720 provided at a proximal end portion of the main body 710, and a holding portion 730 provided at a distal end portion of the main body 710 and removably holding an implant 750 depicted in FIGS. 13A and 13B thereon.

The treatment tool 700 is configured such that, if the main body 710 is inserted into the groove 194B through the proximal end side opening 192B, then the stopper 720 is brought into abutment with the proximal end side opening 192B to restrict further insertion of the main body 710 as depicted in FIG. 12B. In the state in which the stopper 720 abuts with the proximal end side opening 192B, the holding portion 730 projects through the distal end side opening 191B of the guide tool 190B. Consequently, excessive projection of the treatment tool 700 from the guide tool 190B can be prevented, and treatment for a less active tissue can be carried out relatively safely and with relative certainty.

As depicted in FIG. 13A, the implant 750 can include an elongated implant main body (elongated member) 751 and a pair of anchors 752 and 753 provided on the implant main body 751. The implant main body 751 extends through hole portions 752b and 753b provided in the anchors 752 and 753, respectively. The hole portions 752b and 753b preferably have holes of a size and a surface shape with which the implant main body 751 is less likely to slip. Alternatively, the implant main body 751 can be fixed by tying the implant main body 751 to the hole portions 752b and 753b. Such an implant 750 as just described is configured such that the anchors 752 and 753 are held by the holding portion 730 as depicted in FIG. 13B.

The implant main body 751 has a form of a net and has a generally belt-like shape. The implant main body 751 can be configured from an article formed by crossing linear objects with each other into a braid (lattice shape), namely, from a net-like braid. The linear objects may be those having a circular transverse sectional shape, or those having a flattened transverse sectional shape, namely, those of a belt-like shape (ribbon shape). The constituent material of the implant main body 751 is not limited particularly, and, for example, various resin materials, fibers and so forth having biocompatibility such as, for example, polypropylene can be used.

Further, the anchors 752 and 753 have claws 752a and 753a, respectively, and each of the anchors 752 and 753 is engaged with a less active tissue through hooking of the claws 752a or 753a on the less active tissue. Further, the anchors 752 and 753 are individually movable relative to the implant main body 751 such that the spacing distance between the anchors 752 and 753 (the length of the implant main body 751 between the anchors 752 and 753) can be adjusted and maintained. The constituent material of such anchors 752 and 753 as just described is not limited particularly, and, for example, various resin materials, various metal materials and so forth having biocompatibility can be used.

Now, usage of the medical device 100B is described. It is to be noted that, in the following description, a surgical treatment of vaginal vault prolapse is taken as an example.

The surgical procedure in which the medical device 100B is used can include a step of incising part of a vaginal wall H11 prolapsed from the female genitalia to form an incision portion H111, a step of inserting an insertion tool 110, on which a guide tool 190B is mounted, through the incision portion H111 and positioning the distal end of the insertion tool 110 at a sacrospinous ligament H2, a step of pulling out the insertion tool 110 from the guide tool 190B, a step of guiding a treatment tool 700 through the proximal end side opening 192B of the guide tool 190B to the sacrospinous ligament H2 along the groove 194B, a step of carrying out predetermined treatment for the sacrospinous ligament H2 using the treatment tool 700, and a step of correcting the positional displacement of a vagina H1. In the following, description is given in detail.

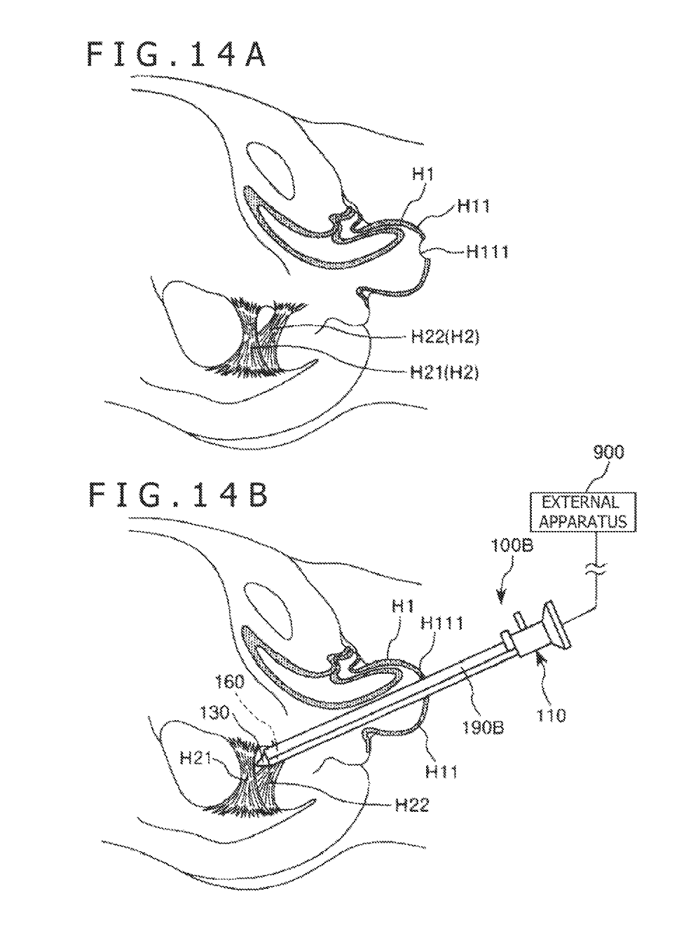

First, the vaginal wall H11 prolapsed from the female genitalia is incised to form the incision portion H111 as depicted in FIG. 14A. Then, the insertion tool 110 connected to the external apparatus 900 and having the guide tool 190B mounted thereon is inserted into the living body through the incision portion H111 and is advanced toward a left side sacrospinous ligament H21 as depicted in FIG. 14B.

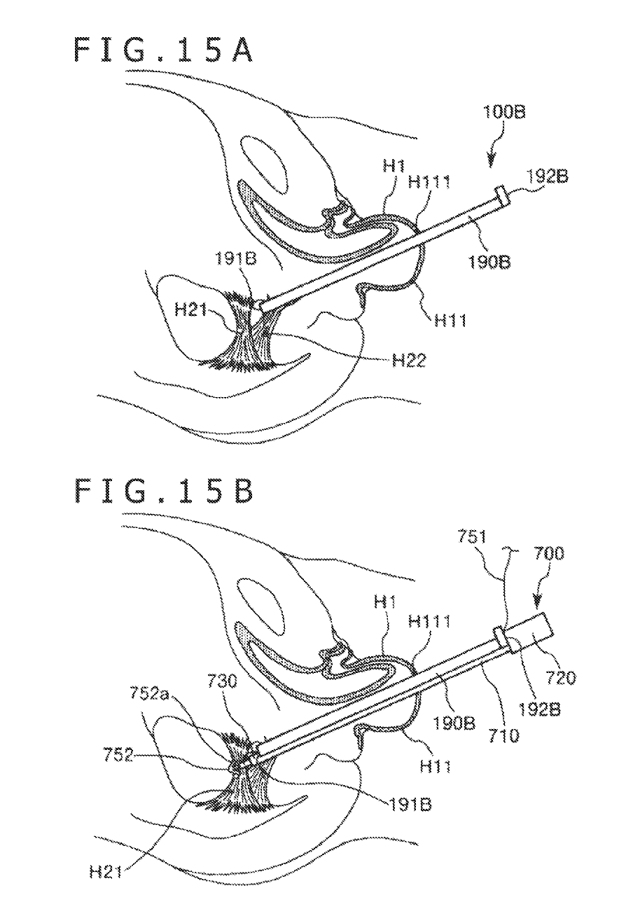

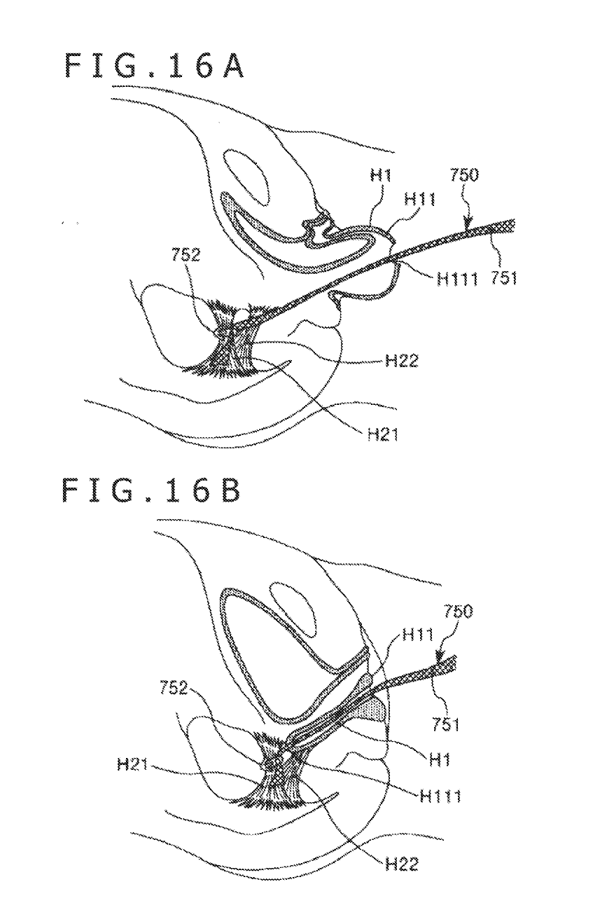

After the distal end of the insertion tool 110 arrives at the sacrospinous ligament H21 or at the proximity of the sacrospinous ligament H21, the insertion tool 110 is pulled out from the guide tool 190B so that the guide tool 190B is indwelled in the living body as depicted in FIG. 15A. Then, a treatment tool 700 having the anchor 752 held on the holding portion 730 thereof is inserted into the living body along the groove 194B of the guide tool 190B. Along with this, if the implant main body 751 is positioned between the main body 710 and the guide tool 190B (in the groove 194B), then the insertion of the treatment tool 700 can be carried out relatively smoothly. If the treatment tool 700 is advanced until the stopper 720 is abutted with the proximal end side opening 192B of the guide tool 190B, then the holding portion 730 projects through the distal end side opening 191B of the guide tool 190B and the claw 752a of the anchor 752 are hooked on the sacrospinous ligament H21. Consequently, the anchor 752 is engaged with the sacrospinous ligament H21. Since the projection amount of the holding portion 730 from the guide tool 190B is controlled by the stopper 720, the anchor 752 can be hooked on the sacrospinous ligament H21 with certainty. Then, the treatment tool 700 and the guide tool 190B are pulled out in order from the living body. Along with this, the anchor 752 is removed from the holding portion 730 and is indwelled in the living body as depicted in FIG. 16A.

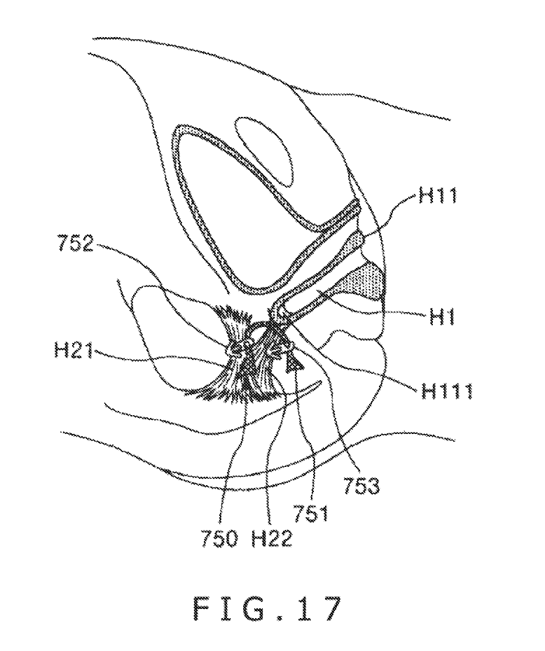

Then, the vagina H1 is returned to its normal position as depicted in FIG. 16B. Then, the anchor 753 is hooked on a right side sacrospinous ligament H22 in accordance with a procedure similar to that of the method described above as depicted in FIG. 17, whereafter the treatment tool 700 and the guide tool 190B are pulled out in order from the living body. Consequently, the implant 750 is placed into a state in which it is indwelled in the living body. Here, the spacing distance between the anchors 752 and 753 and the length of the implant main body 751 positioned between the anchor 752 and the anchor 753 are adjusted in advance so that, when the anchors 752 and 753 are hooked on the sacrospinous ligaments H21 and H22, the vagina H1 is corrected to its normal position. Consequently, if the anchors 752 and 753 are hooked on the left and right sacrospinous ligaments H21 and H22, respectively, then the vaginal wall H11 is lifted by the implant main body 751 and the vagina H1 returns to its normal position. Finally, the incision portion H111 is sutured so that the implant main body 751 is not exposed and then necessary treatments are carried out, thereby ending the surgical procedure.

The medical device 100B of the present exemplary embodiment has been described. It is to be noted that, while, in the medical device 100B, the guide tool 190B is configured from a semi-circular pipe, the shape of the guide tool 190B is not limited particularly only if, in a state in which the anchors 752 and 753 are engaged with the sacrospinous ligaments H21 and H22, respectively, the guide tool 190B can be pulled out from the living body without being caught by the implant main body 751. For example, the guide tool 190B may be configured such that, for example, approximately three fourths of the circumference of a cylinder is removed, or such that it has a cylindrical shape and has a slit extending over an overall rage of an axial direction thereof.

Now, a fourth exemplary example of a medical device is described.

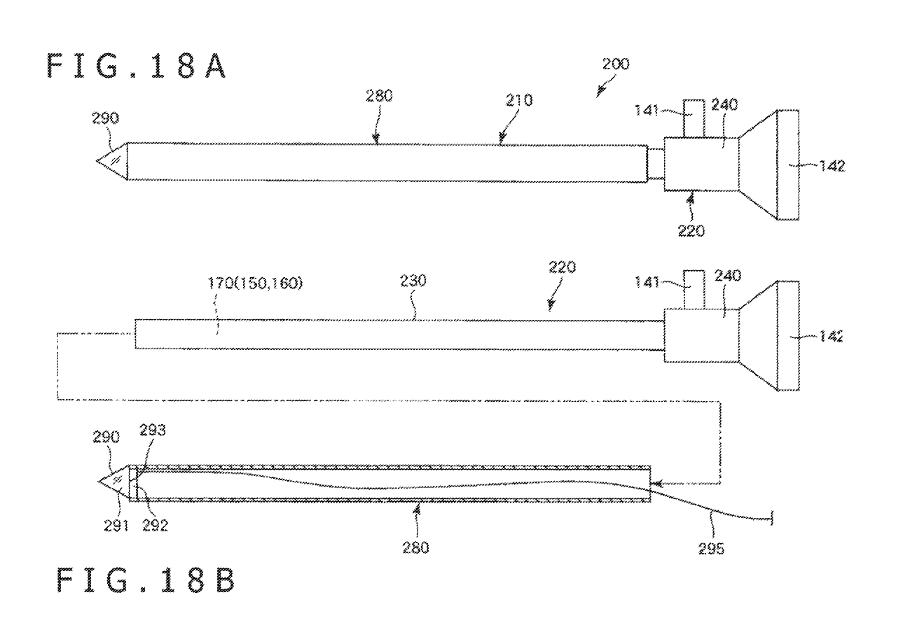

It is to be noted that, in the following description, the left side in FIGS. 18A and 18B is referred to as “distal end” and the right side is referred to as “proximal end” for the convenience of description.

While the medical device of the present exemplary embodiment is described below, description is given principally of differences from the medical device of the first exemplary example while description of similar matters is omitted herein to avoid redundancy.

A medical device 200 depicted in FIGS. 18A and 18B is a device to be used for treatment of pelvic organ prolapse. The medical device 200 is configured from an insertion tool 210 which has an elongated shape and is inserted into a living body. The insertion tool 210 has an insertion tool main body 220, and a mounting portion 280 provided for sliding movement on the insertion tool main body 220 and having an observation portion 290 mounted at a distal end portion thereof. Although the constituent materials of the insertion tool main body 220, the mounting portion 280 and the observation portion 290 are not limited particularly, for example, various resin materials, various metal materials and so forth having biocompatibility can be used.

As depicted in FIG. 18B, the insertion tool main body 220 has an elongated main body 230, and a connector portion 240 provided at a proximal end portion of the main body 230. The main body 230 has a shape extending substantially straightly and has a length of, for example, approximately 10 cm to 30 cm. This provides the insertion tool 210 with a shape suitable for treatment of pelvic organ prolapse. Further, an imaging device 170 is provided in the inside of the main body 230.

The connector portion 240 can include a light source connector 141 and an image signal connector 142. The insertion tool main body 220 can be connected to the external apparatus 900 (refer to FIG. 3) through the light source connector 141 and the image signal connector 142.

The main body 230 has a cylindrical mounting portion 280 provided on an outer periphery thereof so as to be slidably movable with respect to the main body 230. In accordance with an exemplary embodiment, for example, the main body 230 is fitted in the mounting portion 280. Although the inner diameter of the mounting portion 280 is not limited particularly, for example, it preferably is equal to or a little greater than the outer diameter of the main body 230, which makes it possible to suppress looseness of the mounting portion 280 with respect to the main body 230 and improve the operability of the medical device 200. Further, although the outer diameter of the mounting portion 280 is not limited particularly, for example, it preferably is approximately 5 mm to 20 mm, which provides the medical device 200 with a low invasion property and relatively good operability.

The observation portion 290 is removably mounted at a distal end portion of the mounting portion 280. In a state in which the mounting portion 280 is mounted on the insertion tool main body 220, the observation portion 290 is disposed in an opposing relationship to a distal end face of the main body 230. The observation portion 290 is substantially colorless and transparent and has a light permeability. Therefore, illumination light L emitted from the illumination unit 150 passes through the observation portion 290 and can illuminate forwardly of the insertion tool 210. Meanwhile, reflected light L′ which forms an image pickup object image passes through the observation portion 290 and is introduced to the image pickup element 162 (refer to FIG. 3).

The observation portion 290 has a distal end portion 291 of a substantially conical shape having a vertex directed to the distal end side and a proximal end portion 292 of a cylindrical shape extending from the distal end portion 291 to the proximal end side. The observation portion 290 is held by the mounting portion 280 with the proximal end portion 292 thereof inserted in the mounting portion 280. Since the distal end portion 291 tapers, when the insertion tool 210 advances toward a target region in a living body, it exhibits a dissection function of dissecting the biological tissue. Consequently, insertion of the insertion tool 210 into the living body can be carried out relatively smoothly with relative low invasion. It is to be noted that the shape of the distal end portion 291 is not limited to such a conical shape as in the present exemplary embodiment and may be, for example, such a tapering shape (duckbill shape) such the distal end thereof has a linear shape.

Further, the observation portion 290 has a function also as an anchor (indwelled portion) to be indwelled in a living body. On the boundary between the distal end portion 291 and the proximal end portion 292, a step 293 (proximal end face of the distal end portion 291 projecting to the periphery of the proximal end portion 292) is formed. By hooking the step 293 on a less active tissue, the observation portion 290 can be indwelled as an anchor in the living body. Since the observation portion 290 serves also as an anchor in this manner, the device configuration of the medical device 200 is simplified. Further, since the anchor can be operated within the field of view of the image pickup element 162, the anchor can be indwelled accurately in the target region.

Where the observation portion 290 is used as an anchor, it is necessary to remove the observation portion 290 from the mounting portion 280. In order to remove the observation portion 290 from the mounting portion 280, the mounting portion 280 may be slidably moved toward the proximal end side with respect to the main body 230. With such an operation as just described, the observation portion 290 can be removed from the mounting portion 280 relatively simply.

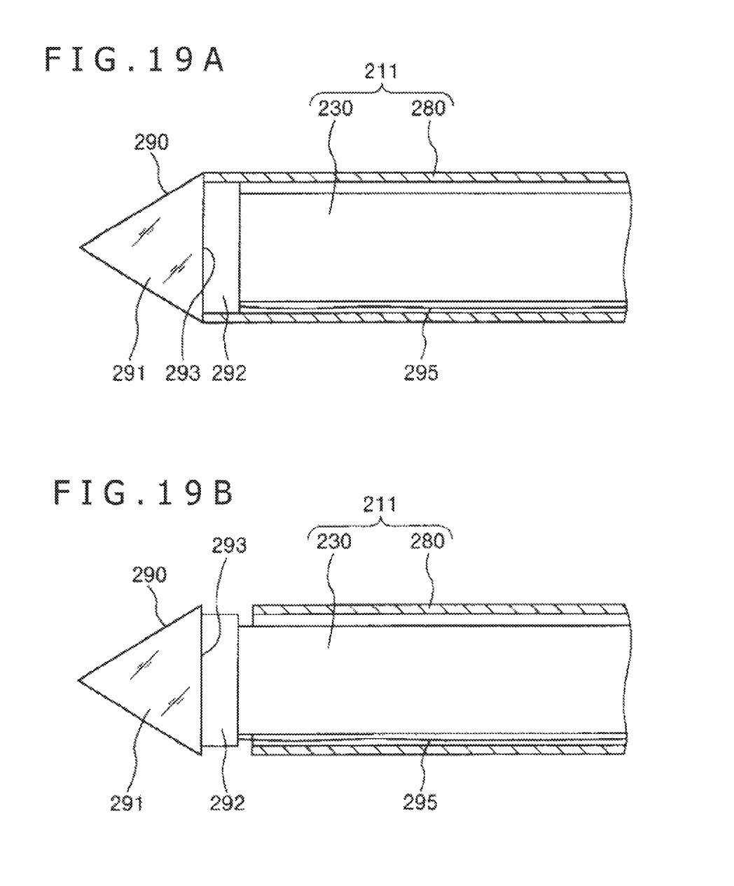

In a state in which the insertion tool 210 is to be inserted into a living body, the observation portion 290 abuts with the distal end face of the main body 230 as depicted in FIG. 19A. Meanwhile, the mounting portion 280 is slidably moved to the proximal end side with respect to the main body 230. Therefore, if, from this state, the mounting portion 280 is slidably moved to the proximal end side with respect to the main body 230, then the observation portion 290 is removed from the mounting portion 280 as depicted in FIG. 19B. In accordance with an exemplary embodiment, for example, by moving the mounting portion 280 with respect to the main body 230, the main body 230 as a pusher urges the observation portion 290 thereby to remove the observation portion 290 from the mounting portion 280. From this, it can be considered that, in the insertion tool 210, the main body (pusher) 230 and the mounting portion 280 configures a removing operation portion 211 for removing the observation portion 290 from the insertion tool 210.

To the observation portion 290 having such a configuration as described above, a string (elongated article) 295 is connected at one end thereof. The string 295 is led out through the proximal end side opening of the mounting portion 280 to the outside passing between the main body 230 and the mounting portion 280. In accordance with an exemplary embodiment, for example, the inside of the mounting portion 280 configures an accommodation space for the string 295. Since the string 295 is accommodated in the accommodation space, when the insertion tool 210 is inserted into a living body, contact between the string 295 and the biological tissue is prevented. Therefore, for example, such a situation that the string 295 is entangled with the biological tissue can be prevented. The string 295 is a suture thread having biocompatibility and is configured from various resin materials having biocompatibility such as polypropylene, fibers and so forth.

It is to be noted that the configuration of the observation portion 290 is not limited to that in the present exemplary embodiment, and, for example, the observation portion 290 may have a claw portion projecting from the distal end portion 291. By the provision of the claw portion, the function as an anchor is improved. Preferably, the claw portion is configured for elastic deformation such that, in a state in which the observation portion 290 is mounted on the mounting portion 280, the claw portion is accommodated in an elastically deformed state in the mounting portion 280, but if the observation portion 290 is dismounted from the mounting portion 280, then the claw portion restores its natural state. Where the claw portion is accommodated in the mounting portion 280, such a situation that advancement of the insertion tool 210 is obstructed by the claw portion is eliminated.

Now, usage of the medical device 200 is described. It is to be noted that, in the following description, a surgical treatment of vaginal vault prolapse is taken as an example.

The medical procedure in which the medical device 200 is used can include a step of incising part of a vaginal wall H11 prolapsed from the female genitalia to form an incision portion H111, a step of inserting an insertion tool 210 through the incision portion H111 and positioning the distal end of the insertion tool 210 at a sacrospinous ligament H2, a step of removing the observation portion 290 from the insertion tool 210 and hooking and engaging the observation portion 290 on and with a sacrospinous ligament H2, a step of pulling out the insertion tool main body 220 and the mounting portion 280 from the living body, and a step of correcting the positional displacement of a vagina H1 using the string 295. In the following, description is given particularly.

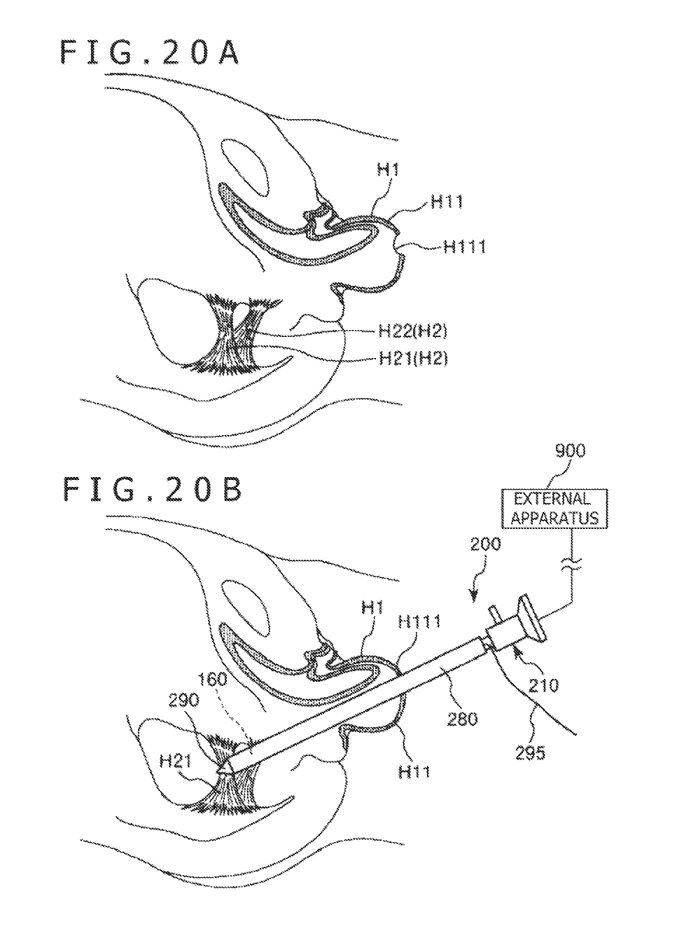

First, the vaginal wall H11 prolapsed from the female genitalia is incised to form the incision portion H111 as depicted in FIG. 20A. Then, the insertion tool 210 connected to the external apparatus 900 is inserted into the living body through the incision portion H111 and advanced toward a left side sacrospinous ligament H21 as depicted in FIG. 20B. Since the observation portion 290 has a function of dissecting a biological tissue, the insertion tool 210 can be advanced smoothly. Further, since an image picked up by the image pickup unit 160 can be confirmed on the real time basis on the monitor, the insertion tool 210 can be advanced toward the sacrospinous ligament H21 relatively accurately and safely.

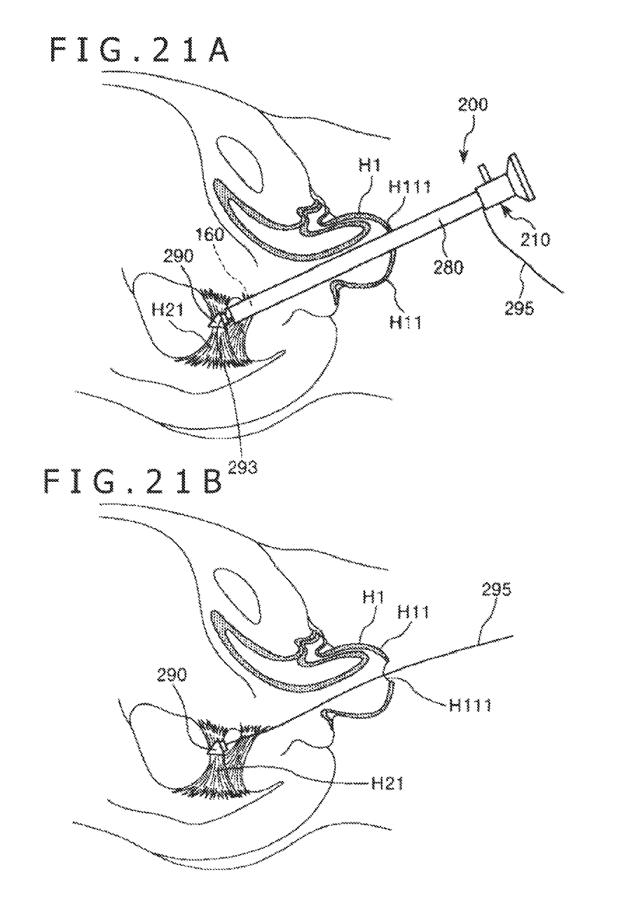

After the distal end of the insertion tool 210 arrives at the sacrospinous ligament H21 or in the proximity of the sacrospinous ligament H21, the mounting portion 280 is slidably moved to the proximal end side with respect to the insertion tool main body 220 or the insertion tool main body 220 is slidably moved to the distal end side with respect to the mounting portion 280 to remove the observation portion 290 from the mounting portion 280 as depicted in FIG. 21A. As a result, the step 293 is exposed in the living body and is caught by and engaged with the sacrospinous ligament H21. Thereafter, the insertion tool main body 220 and the mounting portion 280 are pulled out in order from the living body. Consequently, the observation portion 290 is engaged as an anchor with the sacrospinous ligament H21 and the string 295 connected to the observation portion 290 is exposed to the outside of the body from the incision portion H111 as depicted in FIG. 21B. The hooking of the string 295 on the sacrospinous ligament H21 is completed therewith.

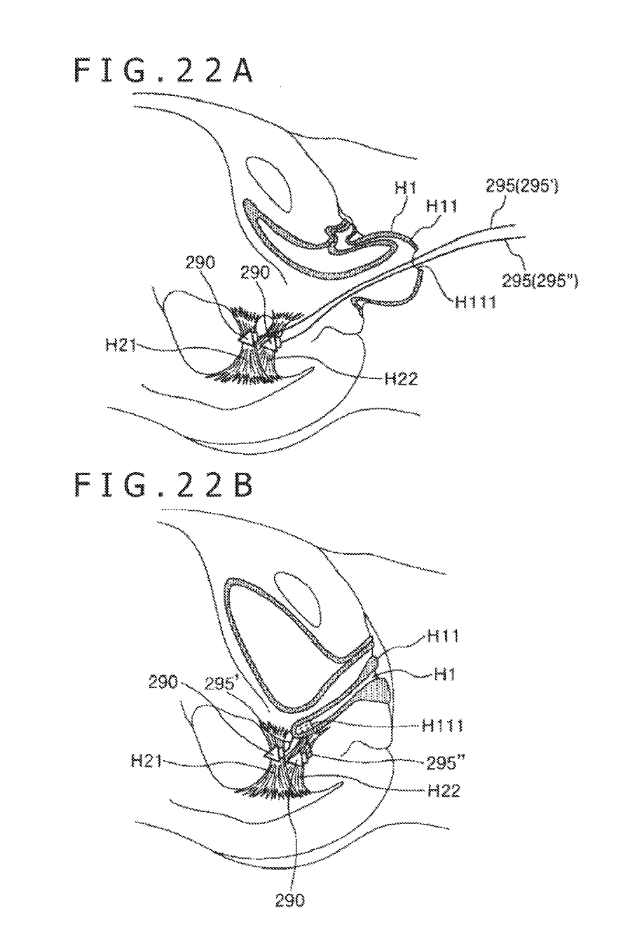

Then, another string 295 is hooked also on a right side sacrospinous ligament H22 by a procedure similar to that of the method described above. Consequently, the opposite ends of the string 295 (295′) hooked on the sacrospinous ligament H21 and the opposite ends of the string 295 (295″) hooked on the sacrospinous ligament H22 are individually exposed to the outside of the body through the incision portion H111 as depicted in FIG. 22A.

Thereafter, the prolapsed vaginal wall H11 is returned to its normal position and the strings 295′ and 295″ are sutured to the vaginal wall H11 so that the state of the vaginal wall H11 may be maintained, whereafter excessive portions of the strings 295′ and 295″ are cut away as depicted in FIG. 22B. Consequently, the sacrospinous ligaments H21 and H22 and the vaginal wall H11 are connected to each other by the strings 295′ and 295″ and the vagina H1 is corrected to its normal position in the female genitalia. Finally, the incision portion H111 is sutured and necessary treatments are carried out, thereby ending the medical procedure.

In this manner, with the medical device 200, since insertion of the insertion tool 210 can be visibly carried out, the insertion tool 210 can arrive at the sacrospinous ligament H2 relatively safely and with relative certainty. Therefore, the observation portion 290 as an anchor can be indwelled accurately in the living body, and the positional displacement of the prolapsed organ can be corrected to return the prolapsed organ to its normal position safely and with certainty.

It is to be noted that, while, in the surgical procedure described above, the strings 295 are hooked on the left and right sacrospinous ligaments H21 and H22, a string 295 may be hooked only on one of the sacrospinous ligaments H21 and H22. Further, while, in the surgical procedure described above, the two strings 295′ and 295″ are sutured to the vaginal wall H11 after they are hooked on the left and right sacrospinous ligaments H21 and H22, the string 295′ may be hooked on the sacrospinous ligament H21 and then sutured to the vaginal wall H11, whereafter the string 295″ is hooked on the sacrospinous ligament H22 and then sutured to the vaginal wall H11.

Now, a first embodiment of the medical device of the present disclosure is described.

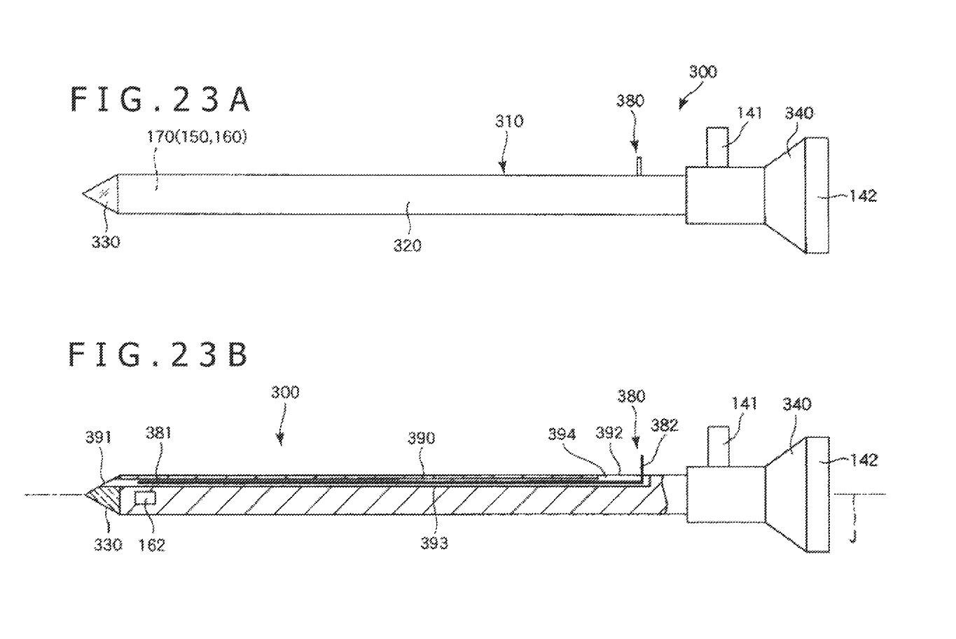

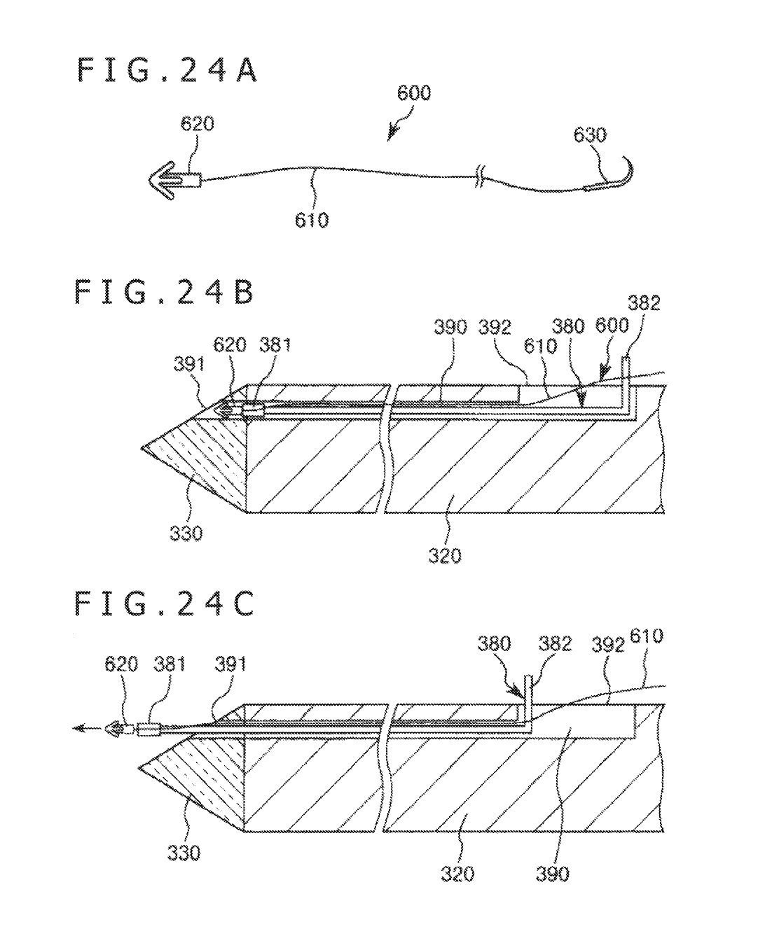

It is to be noted that, in the following description, the left side in FIGS. 23A to 24C is referred to as “distal end” and the right side is referred to as “proximal end” for the convenience of description.

While the medical device of the present embodiment is described below, description is given principally of differences from the medical device of the first exemplary example while description of similar matters is omitted herein to avoid redundancy.

A medical device 300 depicted in FIGS. 23A and 23B is a device to be used for treatment of pelvic organ prolapse. The medical device 300 can include an insertion tool 310 which is in an elongated form and is inserted into a living body, and an operation tool 380 mounted on the insertion tool 310.

The insertion tool 310 can include an elongated main body 320, an observation portion 330 provided at a distal end portion of the main body 320, and a connector portion 340 provided at a proximal end portion of the main body 320. The constituent materials of the main body 320, the observation portion 330 and the connector portion 340 are not limited particularly, and, for example, various resin materials can be used.

The main body 320 has a form of a bar extending straightly. Although the length of the main body 320 is not limited particularly, it is, for example, approximately 10 cm to 30 cm. Further, although the outer diameter of the main body 320 is not limited particularly, it can be, for example, approximately 5 mm to 20 mm, which can provide the insertion tool 310 with a length suitable for treatment of pelvic organ prolapse. Further, an imaging device 170 is provided in the inside of the main body 320.

The connector portion 340 can include a light source connector 141 and an image signal connector 142. The insertion tool 310 can be connected to the external apparatus 900 through the light source connector 141 and the image signal connector 142 (refer to FIG. 3).

The observation portion 330 is provided at the distal end of the main body 320. The observation portion 330 is substantially colorless and transparent and has a light permeability. Further, the observation portion 330 has a substantially conical outer shape having a vertex directed to the distal end side. By forming the observation portion 330 in a tapering shape in this manner, when the insertion tool 310 advances toward a target region in a living body, the observation portion 330 can exhibit a dissection function of dissecting the biological tissue. Consequently, the insertion of the insertion tool 310 into the living body can be carried out relatively smoothly with low invasion. It is to be noted that the shape of the observation portion 330 is not limited to such a conical shape as in the present embodiment and may be, for example, a tapering shape (duckbill shape) such that the distal end of the observation portion 330 has a linear shape. Further, the observation portion 330 is not limited to a colorless transparent one only if it has a light permeability, and may be colored in red, blue, green or the like.

Further, a treatment tool lumen (guide portion) 390 for guiding a treatment tool 600 is formed in the inside of the main body 320 as depicted in FIG. 23B. Further, an operation tool 380 is disposed for sliding movement in the treatment tool lumen 390. It is to be noted that part of the imaging device 170 is omitted for the convenience of illustration.

The treatment tool lumen 390 has an L shape bent at an intermediate portion thereof, and a distal end side opening 391 is positioned at a distal end portion (observation portion 330) of the insertion tool 310 while a proximal end side opening 392 is positioned on a side face of the main body 320. Further, the proximal end side opening 392 is provided such that it is positioned in a living body in a state in which the insertion tool 310 is inserted in the living body.

A distal end portion 393 of the treatment tool lumen 390 which is positioned on the distal end side with respect to the bent portion extends straightly along a center axis J of the insertion tool 310 at a position displaced from the center axis J. Since the distal end portion 393 is disposed in a displaced relationship from the center axis J, the image pickup element 162 can be disposed on the center axis J. Therefore, an image centered at the center axis J can be picked up by the image pickup element 162, and the operability of the insertion tool 310 is improved. Meanwhile, a proximal end portion 394 of the treatment tool lumen 390 which is positioned on the proximal end side with respect to the bent portion has a sufficient length in the axial direction of the main body 320 so that the operation tool 380 can slidably move within the treatment tool lumen 390.

The operation tool 380 is formed from a bar-like member and has an L shape bent at the right angle at an intermediate portion thereof in a corresponding relationship to the shape of the treatment tool lumen 390. A proximal end portion of the operation tool 380 projects to the outside of the main body 320 through the proximal end side opening 392 of the treatment tool lumen 390, and the projecting portion thereof functions as an operation portion 382 for operating the operation tool 380 to slidably move. This facilitates an operation of the operation tool 380. Further, a pusher 381 for pushing out a treatment tool 600 hereinafter described is provided at a distal end portion of the operation tool 380.

If the operation portion 382 is slidably moved to the distal end side with respect to the main body 320, then the treatment tool 600 is pushed to move to the outside (forwardly) of the insertion tool 310 through the distal end side opening 391 by the pusher 381. In this manner, with the operation tool 380, the treatment tool 600 can be pushed out with certainty by a simple operation. In accordance with an exemplary embodiment, for example, since the distal end side opening 391 is positioned at a distal end portion of the insertion tool 310, the treatment tool 600 can be pushed out forwardly of the insertion tool 310 with relative certainty. Further, since the proximal end side opening 392 is positioned outside a living body in a state in which the insertion tool 310 is inserted in the living body, when the insertion tool 310 is used, the operation tool 380 can be operated relatively simply.

The treatment tool 600 is a tool for carrying out treatment for a less active tissue. As depicted in FIG. 24A, the treatment tool 600 is configured from an elongated member including an elongated string (elongated portion) 610, an anchor portion 620 connected to a distal end portion of the string 610, and a needle (suture needle) 630 connected to a proximal end portion of the string 610. The string 610 is a suture needle having biocompatibility and can be configured from various resin materials such as polypropylene, fibers and so forth. Further, the anchor portion 620 can be configured from various resin materials, various metal materials and so forth having biocompatibility.

As depicted in FIG. 24B, the treatment tool 600 is disposed in the treatment tool lumen 390 when the medical device 300 is to be used. In the state in which the treatment tool 600 is disposed in the treatment tool lumen 390, the anchor portion 620 is positioned in front of the pusher 381 at a distal end portion of the treatment tool lumen 390 (in the proximity of the distal end side opening) and the string 610 is exposed through the proximal end side opening of the treatment tool lumen. Then, if the operation tool 380 is slidably moved to the distal end side, then the anchor portion 620 is pushed out forwardly from the insertion tool 310 as depicted in FIG. 24C.

Now, usage of the medical device 300 is described. It is to be noted that, in the following description, a surgical treatment of vaginal vault prolapse is taken as an example.

A medical procedure in which the medical device 300 is used can include a step of incising part of a vaginal wall H11 prolapsed from the female genitalia to form an incision portion H111, a step of inserting an insertion tool 310 on which a treatment tool 600 is mounted through the incision portion H111 and positioning the distal end of the insertion tool 310 at a sacrospinous ligament H2, a step of operating the operation tool 380 to push out the treatment tool 600 toward the sacrospinous ligament H2 and carrying out predetermined treatment for the sacrospinous ligament H2, and a step of correcting the positional displacement of a vagina H1. In the following, description is given particularly.

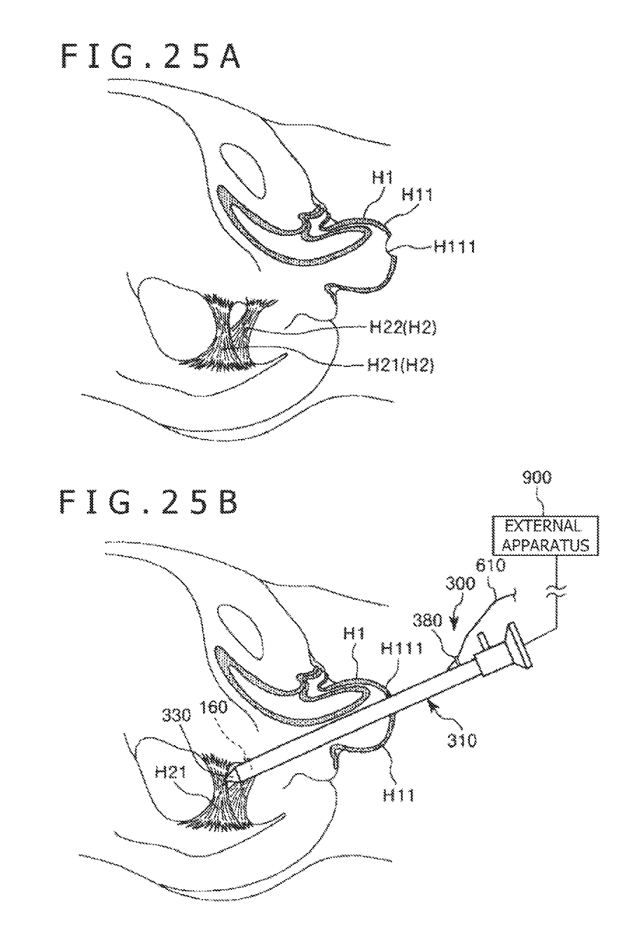

First, the vaginal wall H11 prolapsed from the female genitalia is incised to form the incision portion H111 as depicted in FIG. 25A. Then, the insertion tool 310 connected to the external apparatus 900 and having the treatment tool 600 mounted in the treatment tool lumen 390 thereof is inserted into the living body through the incision portion H111 and advanced toward a left side sacrospinous ligament H21 as depicted in FIG. 25B.

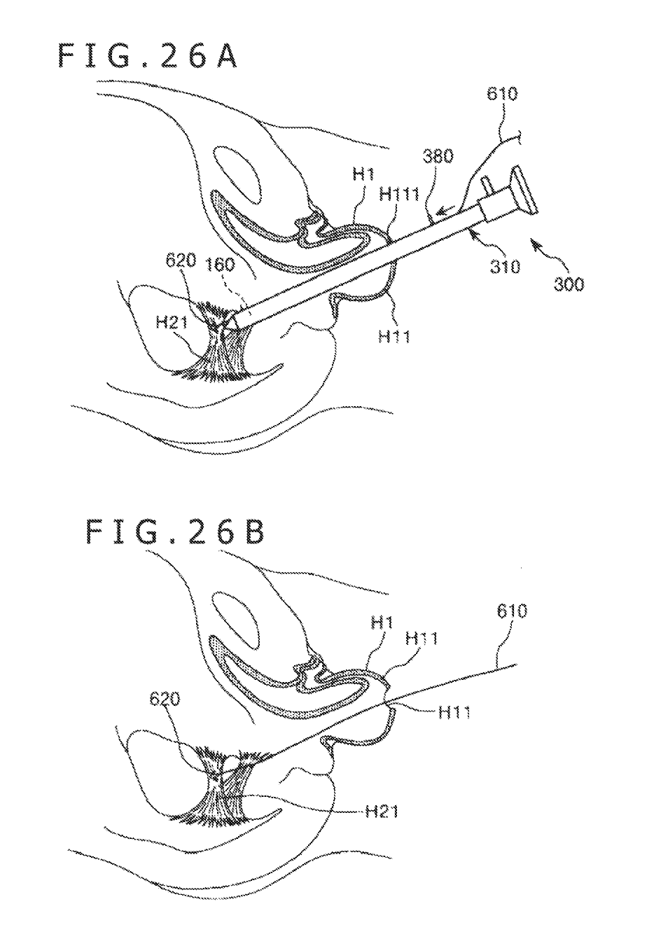

After the distal end of the insertion tool 310 arrives at the sacrospinous ligament H21 or in the proximity of the sacrospinous ligament H21, the treatment tool 600 is guided to the sacrospinous ligament H21 or to the proximity of the sacrospinous ligament H21. Thereafter, the operation tool 380 is operated to slidably move to the distal end side to push out the anchor portion 620 from the insertion tool 310 as depicted in FIG. 26A. The pushed out anchor portion 620 is hooked on and engaged with the sacrospinous ligament H21. Along with this, an image displayed on the monitor may be observed or a string 610 may be drawn to confirm the engagement state of the anchor portion 620 with the sacrospinous ligament H21. Then, the insertion tool 310 is pulled out from the living body. By the process described, the hooking of the string 610 on the left side sacrospinous ligament H21 is completed as depicted in FIG. 26B.

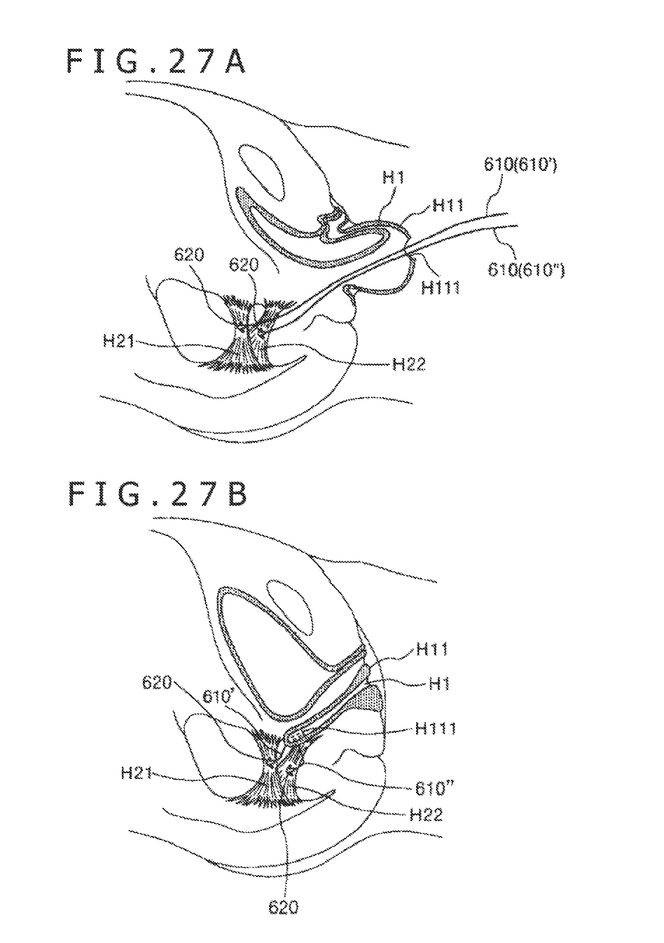

Then, another string 610 is hooked also on a right side sacrospinous ligament H22 by a procedure similar to that of the method described above. Consequently, the proximal end of the string 610 (610′) hooked on the sacrospinous ligament H21 and the proximal end of the string 610 (610″) hooked on the sacrospinous ligament H22 are exposed to the outside of the body through the incision portion H111 as depicted in FIG. 27A.

Then, the prolapsed vaginal wall H11 is returned to its normal position as depicted in FIG. 27B, and the strings 610′ and 610″ are sutured to the vaginal wall H11 using the needles 630 connected to a proximal end portion thereof so that the state of the vaginal wall H11 may be maintained. Then, excessive portions of the strings 610′ and 610″ are cut away. As a result, the sacrospinous ligaments H21 and H22 and the vaginal wall H11 are connected to each other through the strings 610′ and 610″, respectively, and the vagina H1 is corrected to its normal position in the female genitalia. Here, in the treatment tool 600, since the strings 630 are connected to the strings 610 from the beginning, a work for connecting a needle to the string 610 halfway of the medical procedure can be omitted and the medical procedure can be carried out relatively smoothly. Finally, the incision portion H111 is sutured and necessary treatments can be carried out, thereby ending the medical procedure.

In this manner, with the medical device 300, since insertion of the insertion tool 310 can be visibly carried out, the insertion tool 310 can be operated to arrive at the sacrospinous ligament H2 relatively safely and with certainty. Therefore, the anchor portion 620 can be indwelled relatively accurately in the living body, and the positional displacement of the prolapsed organ can be corrected safely and with certainty.

It is to be noted that, while, in the medical procedure described above, the string 610 is hooked on each of the left and right sacrospinous ligaments H21 and H22, the string 610 may be hooked only on one of the sacrospinous ligaments H21 and H22. Further, while, in the medical procedure described above, the two strings 610′ and 610″ are sutured to the vaginal wall H11 after they are hooked on the left and right sacrospinous ligaments H21 and H22, the string 610′ may be hooked on the sacrospinous ligament H21 and then sutured to the vaginal wall H11, whereafter the string 610″ is hooked on the sacrospinous ligament H22 and sutured to the vaginal wall H11.

Further, while, in the present exemplary embodiment, a treatment tool formed by connecting the needle 630 to the string 610 is used as the treatment tool, the needle 630 may be omitted. Thus, the needle 630 may be connected to the string 610 at a suitable stage during the medical procedure (at the stage at which the string 610 is sutured to the vaginal wall).

Now, a medical device according to a second embodiment of the present disclosure is described.

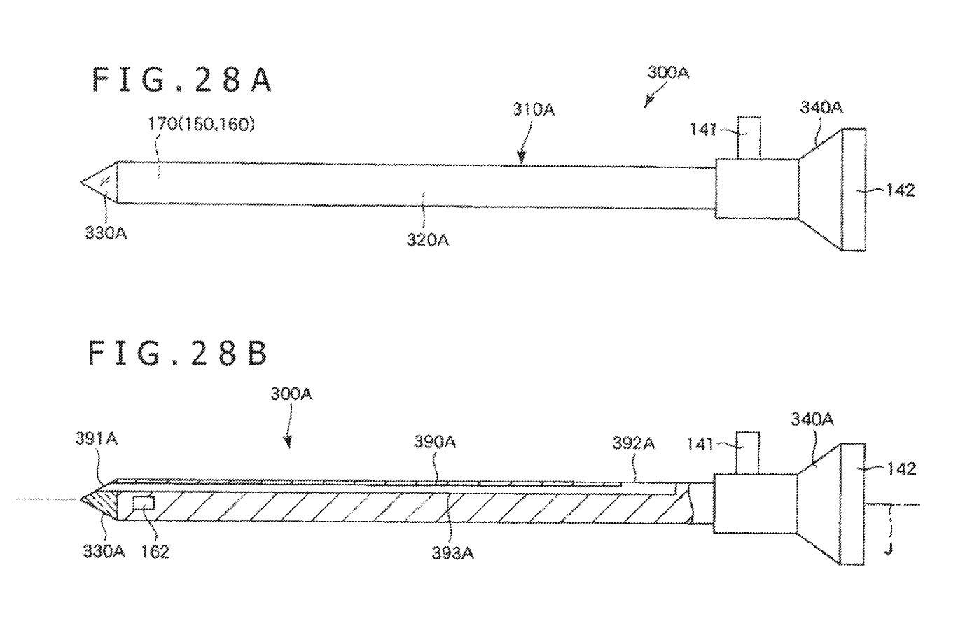

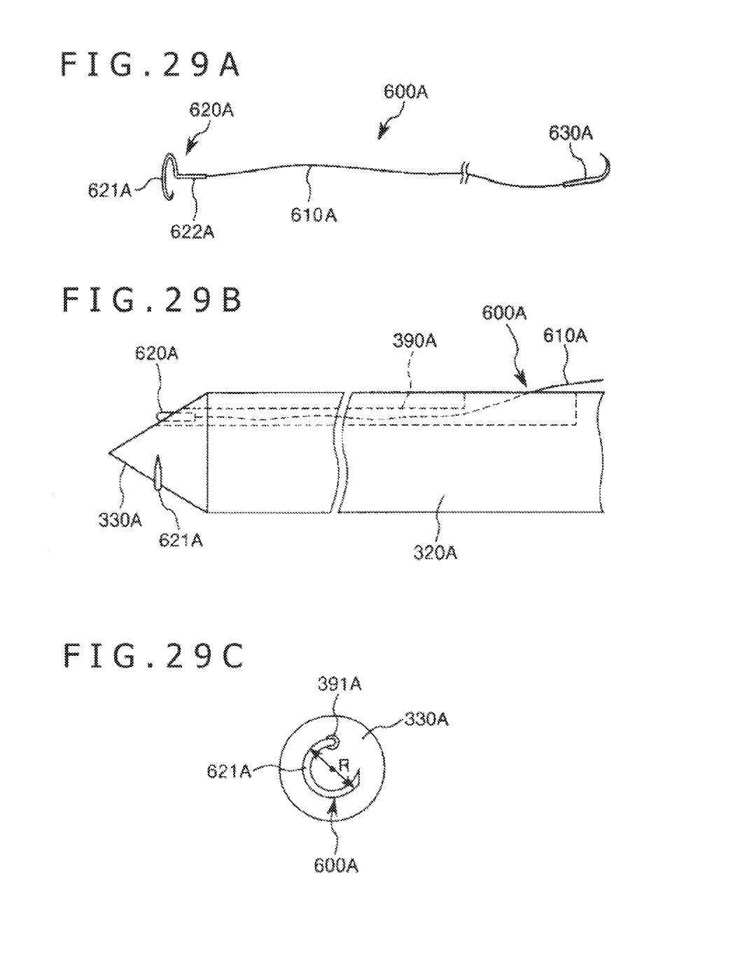

It is to be noted that, in the following description, the left side in FIGS. 28A to 29C is referred to as “distal end” and the right side is referred to as “proximal end” for the convenience of description.

While the medical device of the present embodiment is described below, description is given principally of differences from the medical device of the first embodiment while description of similar matters is omitted herein to avoid redundancy.

A medical device 300A depicted in FIGS. 28A and 28B is a device used for medical treatment of pelvic organ prolapse. The medical device 300A can include an insertion tool 310A of an elongated form which is inserted into a living body.

The insertion tool 310A has an elongated main body 320A, an observation portion 330A provided at a distal end portion of the main body 320A, and a connector portion 340A provided at a proximal end portion of the main body 320A. The constituent materials of the observation portion 330A and the connector portion 340A are not limited particularly, and for example, various resin materials can be used.

The main body 320A has a form of a bar extending substantially straightly. The main body 320A has a length of, for example, approximately 10 cm to 30 cm. Further, the main body 320A has an outer diameter of, for example, approximately to 5 mm to 20 mm. Thus, the insertion tool 310A has a length suitable for treatment of pelvic organ prolapse.

The observation portion 330A is provided at the distal end of the main body 320A. The observation portion 330A is substantially colorless and transparent and has a light permeability. Further, the observation portion 330A has an outer shape of a substantially conical shape having a vertex directed to the distal end side. Further, an imaging device 170 is disposed in the inside of the main body 320A.

A light source connector 141 and an image signal connector 142 can be provided on the connector portion 340A. The insertion tool 310A can be connected to the external apparatus 900 through the light source connector 141 and the image signal connector 142 (refer to FIG. 3).

Further, as depicted in FIG. 28B, a treatment tool lumen (guide portion) 390A for guiding a treatment tool 600A is formed in the inside of the main body 320A as depicted in FIG. 28B. The treatment tool lumen 390A has an L shape bent at the right angle at an intermediate portion thereof, and a distal end side opening 391A is positioned at a distal end portion of the insertion tool 310A (observation portion 330A) while a proximal end side opening 392A is positioned on a side face of the main body 320A. Further, the proximal end side opening 392A is provided such that it is positioned outside a living body in a state in which the insertion tool 310A is inserted in the living body.

A distal end portion 393A of the treatment tool lumen 390A which is positioned on the distal end side with respect to the bent portion extends straightly along a center axis J of the insertion tool 310A at a position displaced from the center axis J. By disposing the distal end portion 393A in a displaced relationship from the center axis J, the image pickup element 162 can be disposed on the center axis J.

The treatment tool 600A is a tool for carrying out treatment for a less active tissue. As depicted in FIG. 29A, the treatment tool 600A is configured from an elongated article having an elongated string (elongated portion) 610A, an anchor portion 620A connected to a distal end portion of the string 610A, and a needle (suture needle) 630A connected to a proximal end portion of the string 610A. The string 610 is a suture string having biocompatibility and can be configured from various resin materials having biocompatibility like polypropylene, fibers and so forth.

Further, the anchor portion 620A is configured by shaping a wire material and has a needle portion 621A curved in an arc and having a sharp needle tip at the distal end thereof, and a stem portion 622A connected to a proximal end portion of the needle portion 621A. The center angle of the arc of the needle portion 621A can be, for example, approximately 120 degrees to 270 degrees. Meanwhile a diameter R of the needle portion 621A is substantially equal to the outer diameter of the observation portion 330A at the place at which the distal end side opening 391A is positioned. Consequently, the needle portion 621A can be disposed along an outer peripheral face of the observation portion 330A as hereinafter described.

Meanwhile, the stem portion 622A has a linear shape and extends in a direction orthogonal to a plane which includes the center axis of the needle portion 621A. The string 610A is connected to a proximal end portion of the stem portion 622A.

The constituent material of the anchor portion 620A is not limited particularly, and various metal materials having biocompatibility such as, for example, stainless steel, aluminum or aluminum alloy, or titanium or titanium alloy can be used.

Such a treatment tool 600A as described above is inserted into the treatment tool lumen 390A as depicted in FIGS. 29B and 29C when the medical device 300A is to be used. Then, while this state is maintained, the treatment tool 600A is inserted into a living body together with the insertion tool 310A. In the insertion state in which the treatment tool 600A is inserted in the treatment tool lumen 390A, the needle portion 621A of the anchor portion 620A projects through the distal end side opening 391A and the string 610A is exposed through the proximal end side opening 392A. By causing the needle portion 621A to project through the distal end side opening 391A through the beginning in this manner, such an operation for pushing out the anchor portion 620A, for example, as in the case of the first embodiment described hereinabove is unnecessary. Therefore, the operation of the medical device 300A is further simplified.

Further, in the insertion state, the needle portion 621A extends along the peripheral direction of an outer peripheral face 331A of the observation portion 330A. In accordance with an exemplary embodiment, for example, the needle portion 621A is shaped along the peripheral direction of the outer peripheral face 331A of the observation portion 330A. By disposing the needle portion 621A in this manner, retraction of the anchor portion 620A into the treatment tool lumen 390A is restricted (prevented). Therefore, treatment for a less active tissue by the treatment tool 600A can be carried out with relative certainty. In addition, excessive projection of the needle portion 621A to the outside of the insertion tool 310A can be prevented, and upon insertion of the insertion tool 310A, the needle portion 621A can be suppressed from damaging the biological tissue. In accordance with an exemplary embodiment, for example, since the needle tip of the needle portion 621A is directed laterally (in a peripheral direction) but is not directed to the distal end side (in the advancing direction of the insertion tool 310A), the effect described above can be exhibited.

It is to be noted that the shape of the needle portion 621A is not limited to that in the present embodiment, and the needle portion 621A may be curved or bent a little or may have a spiral shape extending to the proximal end side such that, for example, in the insertion state, the needle tip thereof is directed to the proximal end side. Even with such shapes, similar effects to those achieved by the present embodiment can be achieved.

Now, usage of the medical device 300A is described. It is to be noted that, in the following description, a surgical treatment of vaginal vault prolapse is taken as an example.

A medical procedure in which the medical device 300A is used can include a step of incising part of a vaginal wall H11 prolapsed from the female genitalia to form an incision portion H111, a step of inserting an insertion tool 310A having a treatment tool 600A mounted thereon through the incision portion H111 and positioning the distal end of the insertion tool 310A at a sacrospinous ligament H2, a step of rotating the insertion tool 310A to carry out predetermined treatment for the sacrospinous ligament H2 using the treatment tool 600A, and a step of correcting the positional displacement of a vagina H1. Detailed description is given below.

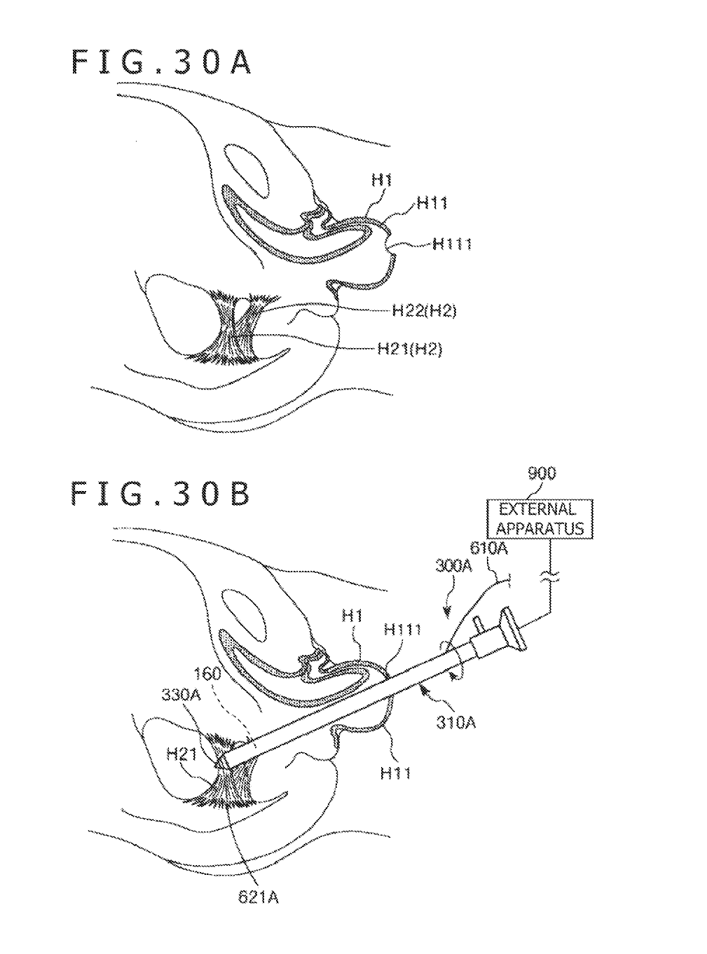

First, the vaginal wall H11 prolapsed from the female genitalia is incised to form the incision portion H111 as depicted in FIG. 30A. Then, the insertion tool 310A connected to the external apparatus 900 and having the treatment tool 600A inserted therein is inserted into the living body through the incision portion H111 and advanced toward a left side sacrospinous ligament H21 as depicted in FIG. 30B.

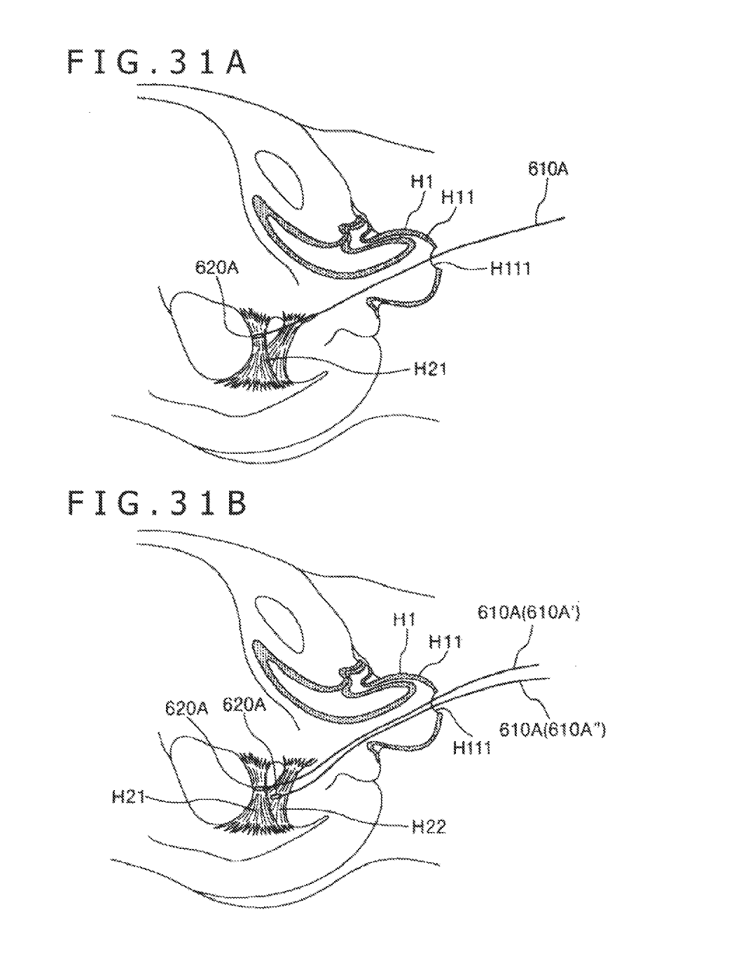

After the distal end of the insertion tool 310A arrives at the sacrospinous ligament H21 or in the proximity of the sacrospinous ligament H21 and the anchor portion 620A is guided to the sacrospinous ligament H21 or to the proximity of the sacrospinous ligament H21, the insertion tool 310A is rotated so that the needle tip of the needle portion 621A advances. Consequently, the needle portion 621A is hooked on the sacrospinous ligament H21 and the anchor portion 620A is engaged with the sacrospinous ligament H21. Since the anchor portion 620A can be engaged with the sacrospinous ligament H21 only by rotating the insertion tool 310A of the medical device 300A in this manner, the medical device 300A has high operability. Along with this, an image displayed on the monitor may be viewed or the string 610A may be pulled to confirm the engagement state of the anchor portion 620A with the sacrospinous ligament H21. Thereafter, the insertion tool 310A is pulled out from the living body. By the procedure described, the anchor portion 620A is indwelled in the living body as depicted in FIG. 31A and the hooking of the string 610A on the left side sacrospinous ligament H21 is completed.

Then, another string 610A is hooked also on a right side sacrospinous ligament H22 by a procedure similar to that of the method described above. Consequently, the proximal end of the string 610A (610A′) hooked on the sacrospinous ligament H21 and the proximal end of the string 610A (610″) hooked on the sacrospinous ligament H22 are exposed to the outside of the body through the incision portion H111 as depicted in FIG. 31B.

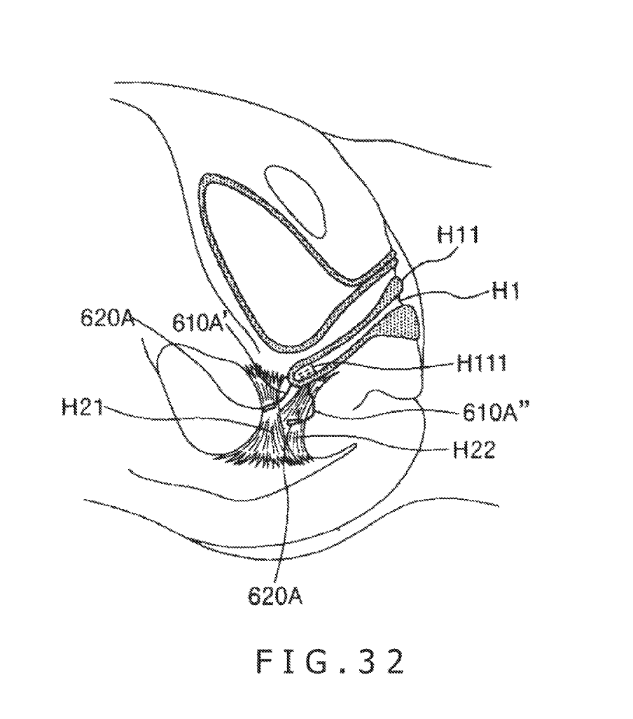

Thereafter, the prolapsed vaginal wall H11 is returned to its normal position and the strings 610′ and 610″ are sutured to the vaginal wall H11 using the needles 630A connected to a proximal end portion thereof, and then excessive portions of the strings 610′ and 610″ are cut away as depicted in FIG. 32. Consequently, the sacrospinous ligaments H21 and H22 and the vaginal wall H11 are connected to each other through the strings 610′ and 610″, respectively, and the vagina H1 is corrected to its normal position in the female genitalia. With the treatment tool 600A, since the needle 630A is connected to the string 610A from the beginning, a work for connecting a needle to the string 610A halfway of the medical procedure can be omitted, and the medical procedure can be carried out relatively smoothly. Finally, the incision portion H111 is sutured and necessary treatments can be carried out, thereby ending the medical procedure.

In this manner, with the medical device 300A, since insertion of the insertion tool 310A can be visibly carried out, the insertion tool 310A can be operated to arrive at the sacrospinous ligament H2 relatively safely and with certainty. Therefore, the anchor portion 620A can be indwelled in the living body accurately, and the positional displacement of the prolapsed organ can be corrected safely and with certainty.

It is to be noted that, while, in the medical procedure described above, the string 610A is hooked on both of the left and right sacrospinous ligaments H21 and H22, the string 610A may be hooked on only one of the sacrospinous ligaments H21 and H22. Further, while, in the medical procedure described above, the two strings 610A′ and 610A″ are sutured to the vaginal wall H11 after they are hooked on the left and right sacrospinous ligaments H21 and H22, the string 610A′ may be hooked on the sacrospinous ligament H21 and then sutured to the vaginal wall H11, whereafter the string 610A″ is hooked on the sacrospinous ligament H22 and sutured to the vaginal wall H11.

Further, while, in the present exemplary embodiment, a treatment tool formed by connecting the needle 630A to the string 610A is used as the treatment tool, the needle 630A may be omitted. Thus, the needle 630A may be connected to the string 610A at a suitable stage during the medical procedure (at the stage at which the string 610 is sutured to the vaginal wall).

Now, a medical device according to a third embodiment of the present disclosure is described.

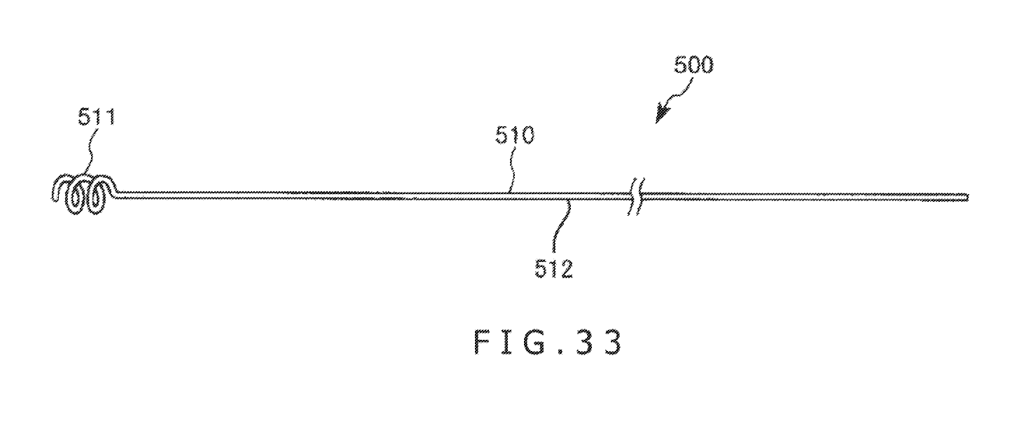

It is to be noted that, in the following description, the left side in FIG. 33 is referred to as “distal end” and the right side is referred to as “proximal end” for the convenience of description.

While the medical device of the present embodiment is described below, description is given principally of differences from the medical device of the second embodiment while description of similar matters is omitted herein to avoid redundancy.

The medical device of the present embodiment is similar to the medical device of the second embodiment except that it is difference only in the treatment tool to be guided.

As depicted in FIG. 33, the treatment tool 500 is a guide wire 510. The guide wire 510 can include an elongated main body (elongated portion) 512 and a distal end portion 511 provided at a distal end portion of the main body 512. The distal end portion 511 is shaped in a coil shape (spiral shape). The distal end portion 511 is deformable into a substantially linear shape by application of external force and returns to its natural state if the application of external force is canceled. It is to be noted that the shape of the distal end portion 511 is not limited particularly if the distal end portion 511 can engage with a less active tissue and may be, for example, an irregularly curved or bent shape. Such a distal end portion 511 as just described functions as an anchor portion which is engaged with a less active tissue. Although the constituent material of the guide wire 510 is not limited particularly, various alloys such as, for example, Ni—Ti-based alloys, Cu—Zn-based alloys and NiAl-based alloys can be applied.

Now, usage of a medical device 300A is described. It is to be noted that, in the following description, a surgical treatment of vaginal vault prolapse is taken as an example.

A medical procedure in which the medical device 300A is used can include a step of incising part of a vaginal wall H11 prolapsed from the female genitalia to form an incision portion H111, a step of inserting an insertion tool 310A through the incision portion H111 and positioning the distal end of the insertion tool 310A at a sacrospinous ligament H2, and a step of inserting a guide wire 510 into a treatment tool lumen 390A of the insertion tool 310A to carry out predetermined treatment for the sacrospinous ligament H2. Detailed description is given below.

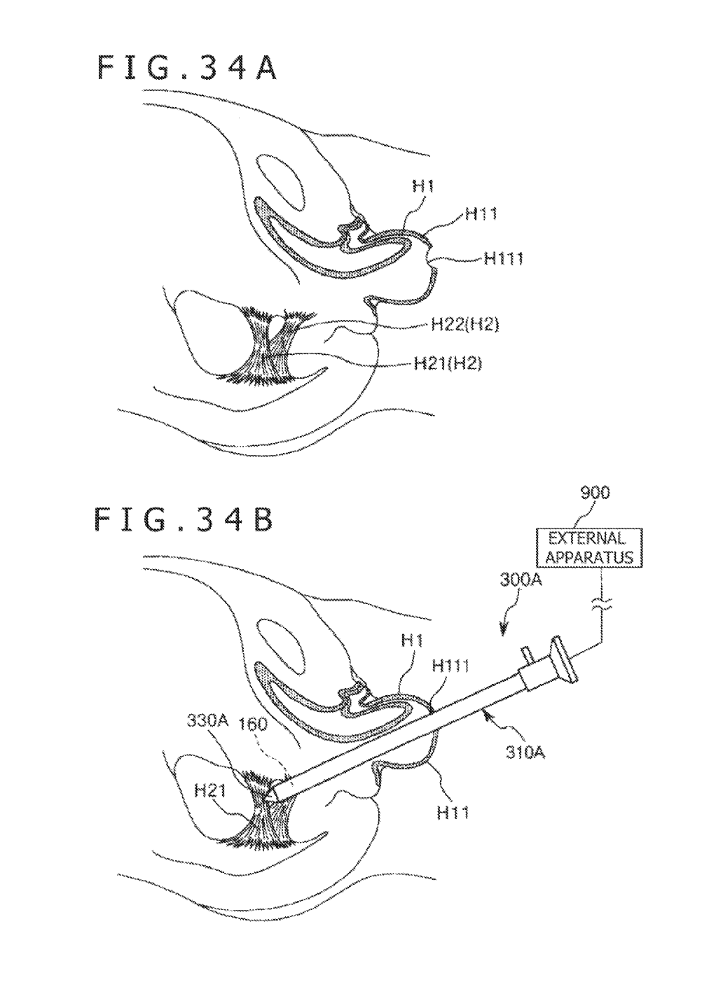

First, the vaginal wall H11 prolapsed from the female genitalia is incised to form the incision portion H111 as depicted in FIG. 34A. Then, the insertion tool 310A connected to an external apparatus 900 and having a treatment tool 600A inserted therein is inserted into the living body through the incision portion H111 and advanced toward a left side sacrospinous ligament H21 as depicted in FIG. 34B.

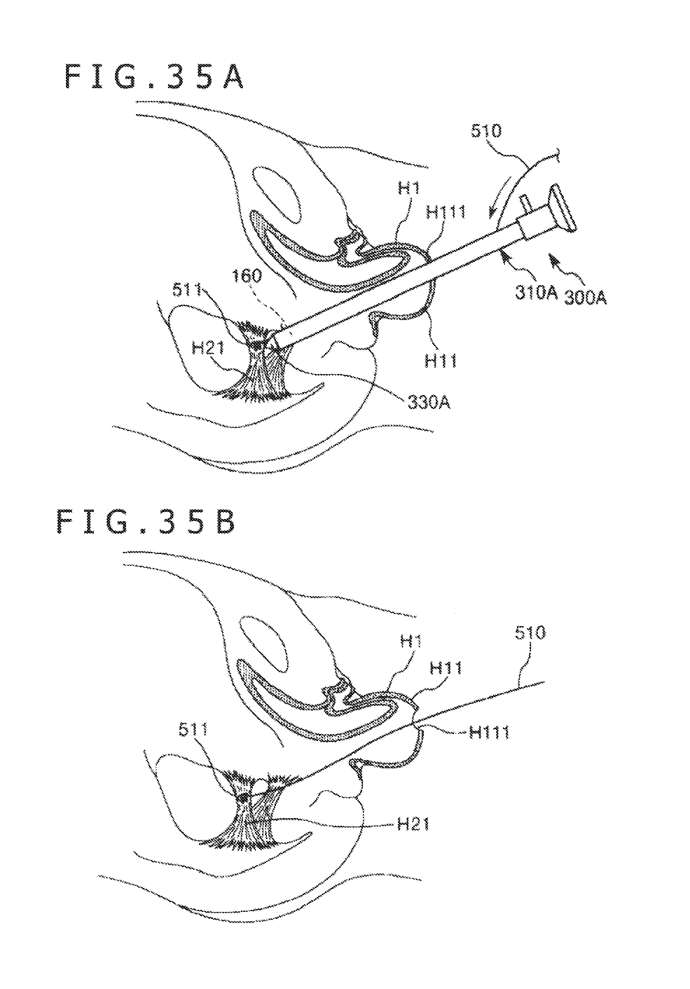

After the distal end of the insertion tool 310A arrives at or in the proximity of the sacrospinous ligament H21, the guide wire 510 is inserted through a proximal end side opening 392A of the treatment tool lumen 390A until the distal end portion 511 of the guide wire 510 projects through a distal end side opening 391A as depicted in FIG. 35A. In accordance with an exemplary embodiment, the guide wire 510 is guided to the sacrospinous ligament H21 by the treatment tool lumen 390A. It is to be noted that the distal end portion 511 passes in a substantially linearly deformed state through the treatment tool lumen 390A. The distal end portion 511 projecting through the distal end side opening 391A is deformed so as to restore its natural state and is hooked on and engaged with the sacrospinous ligament H21 by the deformation. Then, treatment for the sacrospinous ligament H21 by the guide wire 510 is carried out. Thereafter, the insertion tool 310A is pulled out from the living body. By the procedure, the guide wire 510 engaged with the sacrospinous ligament H21 is disposed in the living body as depicted in FIG. 35B.

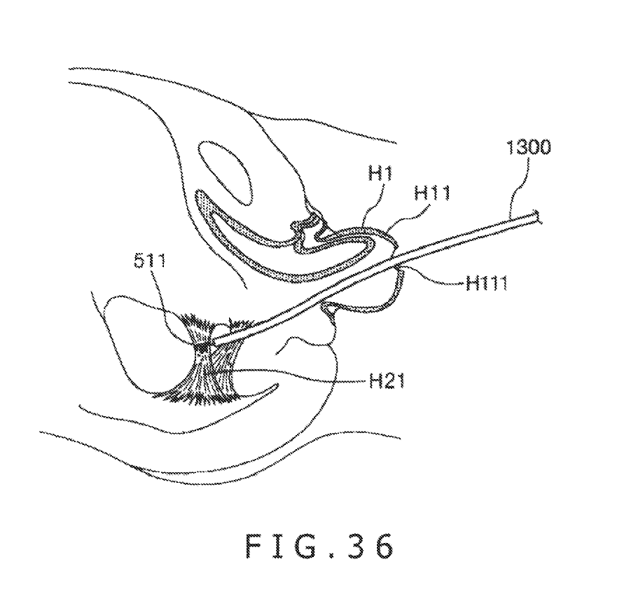

Then, the distal end of a catheter 1300 is guided to the sacrospinous ligament H21 along the guide wire 510 as depicted in FIG. 36. Consequently, treatment for the sacrospinous ligament H21 can be carried out using the catheter 1300. It is to be noted that, also for a right side sacrospinous ligament H22, predetermined treatment can be carried out similarly using the catheter 1300.

Although the medical device of the present disclosure has been described in connection with the embodiments depicted in the drawings, the present disclosure is not limited to the embodiments, but the components of the medical device can be replaced with those of arbitrary configurations which can exhibit similar functions. Further, an arbitrary component may be provided additionally. Further, the medical device of the present disclosure may be a combination of two or more arbitrary ones of the configurations (features) of the embodiments described above.

According to the present disclosure, a medical device which connects an overactive tissue suffering from positional displacement in a living body to a less active tissue having a range of movement smaller than that of the overactive tissue to correct the positional displacement can include an elongated insertion tool including an observation portion configured to observe a distal end side of the medical device using an imaging device, and a guide portion configured to guide a treatment tool, which is to be used to carry out treatment for the less active tissue, to proximity of the less active tissue, the insertion tool having a dissection function for dissecting a biological tissue when the insertion tool is inserted into the living body from an outside of the living body and advances toward the less active tissue in the living body. Therefore, with the medical device, insertion of the insertion tool can be visibly carried out while an image obtained from the imaging device is observed. Consequently, the guide portion can be disposed accurately at a predetermined position, and treatment for the less active tissue by the treatment tool can be carried out relatively safely and accurately.

Accordingly, the medical device of the present disclosure has industrial applicability.

It is to be noted that the medical device according to the present disclosure can take, for example, the following embodiments.

(1) A medical device which connects an overactive tissue suffering from positional displacement in a living body to a less active tissue having a range of movement smaller than that of the overactive tissue to correct the positional displacement, including: an elongated insertion tool including an observation portion configured to observe a distal end side of the medical device using an imaging device, and a guide portion configured to guide a treatment tool, which is to be used to carry out treatment for the less active tissue, to proximity of the less active tissue, the insertion tool having a dissection function for dissecting a biological tissue when the insertion tool is inserted into the living body from an outside of the living body and advances toward the less active tissue in the living body.

(2) The medical device according to (1), wherein the guide portion is a treatment tool lumen provided in an inside of the insertion tool.

(3) The medical device according to (2), wherein the treatment tool lumen has a distal end side opening which is positioned at a distal end portion of the insertion tool.

(4) The medical device according to (2), wherein the treatment tool lumen has a proximal end side opening which is positioned, in a state in which the insertion tool is inserted in the living body, outside the living body.

(5) The medical device according to (1), wherein the treatment tool has an elongated article which has an elongated portion and an anchor portion provided on the elongated portion and configured to engage with the less active tissue.

(6) The medical device according to (5), wherein the treatment tool further has an operation tool configured to push the anchor portion to an outside of the insertion tool from the treatment tool lumen.

(7) The medical device according to (5), wherein the elongated article advances, in an insertion state in which the elongated article is inserted in the treatment tool lumen, toward the less active tissue together with the insertion tool.

(8) The medical device according to (7), wherein, in the insertion state, the anchor portion projects through the distal end side opening of the treatment tool lumen.

(9) The medical device according to (8), wherein, in the insertion state, retraction of the anchor portion into the treatment tool lumen is restricted.

(10) The medical device according to (7), wherein the anchor portion is a linear article shaped along an outer periphery of the insertion tool.

(11) The medical device according to (7), wherein the anchor portion is engaged with the less active tissue by operating the insertion tool to rotate in a state in which the anchor portion is introduced to the less active tissue.

(12) The medical device according to (6), wherein the operation tool can include a pusher provided in the treatment tool lumen and an operation portion provided outside the treatment tool lumen and configured to operate the pusher.

(13) The medical device according to (5), wherein the elongated portion is a string having biocompatibility.

(14) The medical device according to (13), wherein the string has a needle provided at a proximal end portion thereof.

(15) The medical device according to (5), wherein the treatment tool is a guide wire shaped at a distal end portion thereof.

(16) A medical device which connects an overactive tissue suffering from positional displacement in a living body to a less active tissue having a range of movement smaller than that of the overactive tissue to correct the positional displacement, including: an elongated insertion tool including an observation portion configured to observe a distal end side of the medical device using an imaging device and having a dissection function for dissecting a biological tissue when the insertion tool is inserted into the living body from an outside of the living body and advances toward the less active tissue in the living body; and a guide tool configured to be introduced to proximity of the less active tissue by the insertion tool and guide a treatment tool, which is used to carry out treatment for the less active tissue, to the proximity of the less active tissue.

(17) The medical device according to (16), wherein the observation portion is provided at a distal end portion of the insertion tool.

(18) The medical device according to (16), wherein the observation portion tapers toward the distal end thereof and has the dissection function.

(19) The medical device according to (16), wherein the guide tool advances, in a state in which the guide tool is mounted on the insertion tool, toward the less active tissue together with the insertion tool.

(20) The medical device according to (19), wherein the guide tool has an elongated shape and has a through-hole or a groove which connects one end and the other end of the guide tool, and the guide tool is mounted on the insertion tool through the through-hole or the groove and guides the treatment tool to the less active tissue.

(21) The medical device according to (20), wherein the guide tool has a form of a pipe having the through-hole therein, and the guide tool is inserted into the living body in a state in which the insertion tool is fitted in the guide tool.

(22) The medical device according to (16), wherein the treatment tool is a tool engaging an elongated article with the less active tissue.

(23) The medical device according to (22), wherein the elongated article is a string or a net having biocompatibility.

(24) A medical device which connects an overactive tissue suffering from positional displacement in a living body to a less active tissue having a range of movement smaller than that of the overactive tissue to correct the positional displacement, including: an elongated insertion tool including an observation portion configured to observe a distal end side of the medical device using an imaging device, and an indwelled portion configured to be indwelled in the living body in a state in which the indwelled portion engages with the less active tissue; and the insertion tool being inserted into the living body from the outside of the living body.

(25) The medical device according to (24), wherein the observation portion is provided at a distal end portion of the insertion tool.

(26) The medical device according to (24), wherein the observation portion serves also as the indwelled portion.

(27) The medical device according to (24), wherein the insertion tool has a disengaging operation portion configured to disengage the indwelled portion from the insertion tool.

(28) The medical device according to (27), wherein the disengaging operation portion can include a mounting portion on which the indwelled portion is mounted, and a pusher configured to move relative to the mounting portion to urge the indwelled portion to disengage the indwelled portion from the mounting portion.

(29) The medical device according to (28), wherein the mounting portion has a form of a pipe, and the pusher is inserted in an inside of the mounting portion.

(30) The medical device according to (24), wherein the indwelled portion has connected thereto an elongated article which connects the overactive tissue and the less active tissue to each other.

(31) The medical device according to (30), wherein the elongated article is a string or a net having biocompatibility.

(32) The medical device according to (30), wherein the insertion tool has an accommodation space in which the elongated article is accommodated.

(33) The medical device according to any one of (1), (16), and (24), wherein the medical device is used for treatment of pelvic organ prolapse.

(34) The medical device according to any one of (1), (16), and (24), wherein the overactive tissue is bladder, urethra, uterus, vagina, small intestine, large intestine, colon or rectum.

(35) The medical device according to any one of (1), (16), and (24), wherein the less active tissue is bone, tendon, ligament, fascia or muscle tissue.

The medical device according to (1) to (15) described above can include elongated insertion tool including an observation portion configured to observe a distal end side of the medical device using an imaging device, and a guide portion configured to guide a treatment tool, which is to be used to carry out treatment for a less active tissue, to the proximity of the less active tissue, the insertion tool having a dissection function for dissecting a biological tissue when the insertion tool is inserted into the living body from the outside of the living body and advances toward the less active tissue in the living body. Therefore, with the medical device, insertion of the insertion tool can be carried out visibly while an image obtained from the imaging device is observed. Consequently, the guide portion can be disposed accurately at a predetermined position, and treatment for the less active tissue by the treatment tool can be carried out safely and accurately.

The medical device according to (16) to (23) described above can include an elongated insertion tool including an observation portion configured to observe a distal end side of the medical device using an imaging device and having a dissection function for dissecting a biological tissue when the insertion tool is inserted into the living body from the outside of the living body and advances toward a less active tissue in the living body, and a guide tool configured to be introduced to the proximity of the less active tissue by the insertion tool and guide a treatment tool, which is used to carry out treatment for the less active tissue, to the proximity of the less active tissue. Therefore, with the medical device, insertion of the insertion tool can be carried out visibly while an image obtained from the imaging device is observed. Consequently, the guide tool can be disposed accurately at a predetermined position, and treatment for the less active tissue by the treatment tool can be carried out safely and accurately.

The medical device according to (24) to (32) described above can include an elongated insertion tool including an observation portion configured to observe a distal end side of the medical device using an imaging device, and an indwelled portion configured to be indwelled in a living body in a state in which the indwelled portion engages with a less active tissue, the insertion tool being inserted into the living body from the outside of the living body. Therefore, with the medical device, insertion of the insertion tool can be carried out visibly while an image obtained from the imaging device is observed. Consequently, an indwelling tool can be indwelled accurately at a predetermined position, and a manipulation can be carried out safely and accurately.

The detailed description above describes a medical device. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A medical device which connects an overactive tissue suffering from positional displacement in a living body to a less active tissue having a range of movement smaller than that of the overactive tissue to correct the positional displacement, the medical device comprising:
an elongated insertion tool including an observation portion configured to observe a distal end side of the medical device using an imaging device, and a guide portion configured to guide a treatment tool, which is to be used to carry out treatment for the less active tissue, to proximity of the less active tissue, the insertion tool having a dissection function for dissecting a biological tissue when the insertion tool is inserted into the living body from an outside of the living body and advances toward the less active tissue in the living body.

2. The medical device according to claim 1,
wherein the guide portion is a treatment tool lumen in an inside of the insertion tool.

3. The medical device according to claim 2,
wherein the treatment tool lumen has a distal end side opening which is positioned at a distal end portion of the insertion tool.

4. The medical device according to claim 2,
wherein the treatment tool lumen has a proximal end side opening which is positioned, in a state in which the insertion tool is inserted in the living body, outside the living body.

5. The medical device according to claim 1,
wherein the treatment tool has an elongated article which has an elongated portion and an anchor portion on the elongated portion and configured to engage with the less active tissue.

6. The medical device according to claim 5,
wherein the treatment tool further has an operation tool configured to push the anchor portion to an outside of the insertion tool from the treatment tool lumen.

7. The medical device according to claim 6,
wherein the elongated article advances, in an insertion state in which the elongated article is inserted in the treatment tool lumen, toward the less active tissue together with the insertion tool.

8. The medical device according to claim 7,
wherein, in the insertion state, the anchor portion projects through the distal end side opening of the treatment tool lumen.

9. The medical device according to claim 8,
wherein, in the insertion state, retraction of the anchor portion into the treatment tool lumen is restricted.

10. The medical device according to claim 7,
wherein the anchor portion is a linear article shaped along an outer periphery of the insertion tool.

11. The medical device according to claim 7,
wherein the anchor portion is engaged with the less active tissue by operating the insertion tool to rotate in a state in which the anchor portion is introduced to the less active tissue.

12. A medical device which connects an overactive tissue suffering from positional displacement in a living body to a less active tissue having a range of movement smaller than that of the overactive tissue to correct the positional displacement, the medical device comprising:
an elongated insertion tool including an observation portion configured to observe a distal end side of the medical device using an imaging device and having a dissection function for dissecting a biological tissue when the insertion tool is inserted into the living body from an outside of the living body and advances toward the less active tissue in the living body; and
a guide tool configured to be introduced to proximity of the less active tissue by the insertion tool and guide a treatment tool, which is used to carry out treatment for the less active tissue, to the proximity of the less active tissue.

13. The medical device according to claim 12, wherein the observation portion is at a distal end portion of the insertion tool.

14. The medical device according to claim 12, wherein the observation portion tapers toward the distal end thereof and has the dissection function.

15. The medical device according to claim 12, wherein
the guide tool advances, in a state in which the guide tool is mounted on the insertion tool, toward the less active tissue together with the insertion tool; and
the guide tool has an elongated shape and has a through-hole or a groove which connects one end and the other end of the guide tool, and the guide tool is mounted on the insertion tool through the through-hole or the groove and guides the treatment tool to the less active tissue.

16. The medical device according to claim 15, wherein the guide tool has a form of a pipe having the through-hole therein, and
the guide tool is inserted into the living body in a state in which the insertion tool is fitted in the guide tool.

17. A method for treating an overactive tissue suffering from positional displacement in a living body, the method comprising:
incising a portion of a vaginal wall prolapsed from a female genitalia to form an incision portion;
inserting an elongated insertion tool on which a guide tool is mounted, through the incision portion, the elongated insertion tool including an observation portion configured to observe a distal end side of the medical device using an imaging device, and an indwelled portion configured to be indwelled in the living body in a state in which the indwelled portion engages a less active tissue;
positioning a distal end of the insertion tool at the less active tissue;
pulling out the insertion tool from the guide tool;
inserting a treatment tool through a proximal end side opening of the guide tool and guiding the treatment tool to the less active tissue;
carrying out a predetermined treatment for the less active tissue using the treatment tool; and
correcting the positional displacement of the overactive tissue.

18. The method according to claim 17, comprising:
dissecting a biological tissue with the insertion tool when the insertion tool is inserted into the living body from an outside of the living body and advances toward the less active tissue in the living body.

19. The method according to claim 17, wherein the predetermined treatment comprises:
hooking a first string on a sacrospinous ligament with the treatment tool;
pulling the treatment tool from the guide tool;
pulling the guide tool from living body, and wherein opposite ends of the first string hooked on the sacrospinous ligament are placed in a state in which they are exposed to an outside of the living body; and
wherein the correcting of the positional displacement of the overactive tissue is returning a prolapsed vaginal wall to a corrected position.

20. The method according to claim 19, comprising:
hooking of a second string on an opposite side of the sacrospinous ligament with the treatment tool, pulling the treatment from the guide tool, and the pulling the guide tool from the living body; and
cutting excessive portions of the first and second strings.
